(19) United States
(12) Patent Application Publication
(10) Pub. No.: US 2023/0220472 A1
(43) Pub. Date: Jul. 13, 2023

(54) DETERIMINING RISK OF SPONTANEOUS CORONARY ARTERY DISSECTION AND MYOCARDIAL INFARCTION AND SYSEMS AND METHODS OF USE THEREOF

(71) Applicants: The Regents of the University of Michigan, Ann Arbor, MI (US); The University of British Columbia, Vancouver (CA)

(72) Inventors: Santhi Ganesh, Ann Arbor, MI (US); Liam Brunham, Vancouver (CA); Jacqueline Saw, Vancouver (CA)

(21) Appl. No.: 18/000,060

(22) PCT Filed: May 28, 2021
(86) PCT No.: PCT/US21/34780

§ 371 Date: Nov. 28, 2022

Related U.S. Application Data
(60) Provisional application No. 63/031,481, filed on May 28, 2020.

(51) Int. Cl. C12Q 1/6883; A61K 31/616; A61P 9/10

(52) U.S. Cl. CPC C12Q 1/6883; A61K 31/616; A61P 9/10; C12Q 2600/118; C12Q 2600/156

(57) ABSTRACT

Provided herein are systems and methods for determining a subject's risk of spontaneous coronary artery dissection (SCAD) and myocardial infarction (MI) and systems and methods of using SCAD and/or MI risk for treatment thereof.

### STATEMENT REGARDING FEDERAL FUNDING

This invention was made with government support under HL122684 and HL139672 awarded by the National Institutes of Health. The government has certain rights in the invention.

### FIELD

Provided herein are systems and methods for determining a subject's risk of spontaneous coronary artery dissection (SCAD) and myocardial infarction (MI) and systems and methods of using SCAD and/or MI risk for treatment thereof.

### BACKGROUND

Spontaneous coronary artery dissection (SCAD) is an increasingly recognized cause of myocardial infarction (MI) in young and otherwise healthy women, for which the etiology is incompletely understood. SCAD is an important cause of MI in women <50 years of age (ref 1; incorporated by reference in its entirety). SCAD is defined as a non-traumatic, non-iatrogenic and non-atherosclerotic separation of the coronary arterial wall by intramural hemorrhage, most often elicited by spontaneous intimal tear or rupture of vasa vasorum, causing accumulation of intramural hematoma that compresses the true arterial lumen, resulting in compromised coronary artery blood flow and MI (FIG. 6). It is hypothesized that SCAD results from a combination of susceptibility to dissection due to a predisposing arteriopathy that weakens the arterial wall, compounded by an additional precipitating trigger (i.e. emotional or physical stressor) that culminates in the arterial disruption (Refs. 2-3; incorporated by reference in their entireties).

### SUMMARY

Provided herein are systems and methods for determining a subject's risk of spontaneous coronary artery dissection (SCAD) and myocardial infarction (MI) and systems and methods of using SCAD and/or MI risk for treatment thereof.

In some embodiments, provided herein are methods of assessing the risk of spontaneous coronary artery dissection (SCAD) and/or SCAD-related myocardial infarction (MI) in a subject suffering from fibromuscular dysplasia (FMD) comprising: (a) testing a sample from the subject for biomarkers of SCAD; and (b) assessing the subject's risk of SCAD. In some embodiments, the biomarkers of SCAD are selected from rs11207415, rs12740679, rs78377252, rs9349379, rs78349783, rs11172113, and rs28451064. In some embodiments, any combination of two or more of rs11207415, rs12740679, rs78377252, rs9349379, rs78349783, rs11172113, and rs28451064 are analyzed. In some embodiments, assessing the subject's risk of SCAD comprises: (i) calculating a risk score based on the biomarkers for SCAD; and (ii) comparing the risk score to a threshold to determine the subject's risk for SCAD. In some embodiments, the presence of any biomarker is weighted according to its odds ratio in order to calculate the risk score.

In some embodiments, provided herein are methods of preventing spontaneous coronary artery dissection (SCAD) and/or SCAD-related myocardial infarction (MI) in a subject suffering from fibromuscular dysplasia (FMD) comprising: (1) assessing the risk of SCAD by the method of described herein; and (2) administering a prophylactic regime to reduce the subject's risk for SCAD. In some embodiments, the prophylactic regime comprises one or more of: (i) administering aspirin to the subject; (ii) cessation of treatment with triptan medications; (iii) administration of contraception; (iv) avoiding activities that increase arterial strain or spikes in blood pressure; (v) monitoring blood pressure; (vi) administering a beta blocker; and (vii) administering antiplatelet therapy.

Embodiments of the present disclosure include a method of predicting SCAD risk (or SCAD-related MI) in a subject (e.g., a subject suffering from FMD)). In accordance with these embodiments, the method includes quantifying levels of at one or more biomarkers (e.g., single nucleotide polymorphisms (SNPs) from a sample from a subject; calculating a risk score based on the presence/absence of the one or more biomarkers; and determining subject's risk for SCAD. In some embodiments, the subject is assigned a risk level, such as low risk, intermediate risk, or high risk of SCAD based on the calculated risk score. In some embodiments, the biomarkers are weighted in the risk score calculation.

Embodiments of the present disclosure also include a biomarker panel for determining SCAD risk in a subject. In accordance with these embodiments, the panel includes at least two of the following biomarkers: rs11207415, rs12740679, rs78377252, rs9349379, rs78349783, rs11172113, and rs28451064.

In some embodiments, the present disclosure provides a risk score, based on the presence/absence of one or more biomarkers (e.g., SNPs) to determine a subject's risk (e.g., low, intermediate, high, etc.) of SCAD, thereby permitting selection of appropriate therapies to treat the subject. In some embodiments, provided herein are methods of assessing the risk of atherosclerotic coronary artery disease and/or atherosclerotic-related myocardial infarction (MI) in a subject comprising: (a) testing a sample from the subject for biomarkers of that confer inverse risk for atherosclerotic coronary artery disease and/or atherosclerotic-related MI; and (b) assessing the subject's risk of MI. In some embodiments, the biomarkers are selected from rs11207415, rs12740679, rs78377252, rs9349379, rs78349783, rs11172113, and rs28451064. In some embodiments, any combination of two or more of rs11207415, rs12740679, rs78377252, rs9349379, rs78349783, rs11172113, and rs28451064 are analyzed. In some embodiments, assessing the subject's risk of atherosclerotic coronary artery disease and/or atherosclerotic-related MI comprises: (i) calculating a risk score based on the biomarkers; and (ii) comparing the risk score to a threshold to determine the subject's risk for MI. In some embodiments, the presence of any biomarker is weighted according to its odds ratio in order to calculate the risk score.

Embodiments of the present disclosure include a method of predicting risk of atherosclerotic coronary artery disease and/or atherosclerotic-related MI in a subject. In accordance with these embodiments, the method includes quantifying levels of at one or more biomarkers (e.g., single nucleotide polymorphisms (SNPs)) from a sample from a subject; calculating a risk score based on the presence/absence of the one or more biomarkers; and determining subject's risk for atherosclerotic coronary artery disease and/or atherosclerotic-related MI. In some embodiments, the subject is assigned a risk level, such as low risk, intermediate risk, or high risk based on the calculated risk score. In some embodiments, the biomarkers are weighted in the risk score calculation.

Embodiments of the present disclosure also include a biomarker panel for determining risk of atherosclerotic coronary artery disease and/or atherosclerotic-related MI in a subject. In accordance with these embodiments, the panel includes at least two of the following biomarkers: rs11207415, rs12740679, rs78377252, rs9349379, rs78349783, rs11172113, and rs28451064.

In some embodiments, the present disclosure provides a risk score, based on the presence/absence of one or more biomarkers (e.g., SNPs) to determine a subject's risk (e.g., low, intermediate, high, etc.) of atherosclerotic coronary artery disease and/or atherosclerotic-related MI, thereby permitting selection of appropriate therapies to treat the subject.

### BRIEF DESCRIPTION OF THE DRAWINGS

FIG. 1. Discovery study design for the SCAD genome-wide association analysis (GWAS), and PRS_{SCAD} development and testing. In both the discovery and replication phases, CanSCAD study samples were analyzed with control subjects derived from the MGI biorepository with electronic health-record based phenotyping to exclude individuals with vascular diseases or connective tissue disorders. The association of genetic variants with SCAD and MI was tested by means of a PRS developed with the top ranked loci in the GWAS meta-analysis of SCAD discovery and replication analyses (PRS_{SCAD}). The PRS_{SCAD} was tested for association with SCAD and MI in a cohort of individuals with FMD, and for CAD and MI in the UK Biobank and MVP.

FIGS. 2A-B. SCAD GWAS meta-analysis results. FIG. 2A, Manhattan plot and QQ plot for the meta-analysis of SCAD discovery and replication groups (Ncases=433, Ncontrols=8,470). Discovery GWAS and replication association analysis were all based on generalized mixed models in SAIGE, which uses the saddlepoint approximation (SPA) correction that accounts for case and control imbalances. GC correction was applied before standard error weighted meta-analysis. SNPs with imputation Rsq≥0.8 and MAF≥1% were analyzed. P values are two-sided and unadjusted for multiple testing and variants meeting the genome-wide significance Bonferroni corrected threshold (association P<5×10⁻⁸) are shown in blue. All of the association models are adjusted for PCs; and age, sex, PCs-matched between cases and controls. The λ_{GC} value is 0.95. FIG. 2B, Regional association plots with gene annotation for the chromosomes 1q21, 6p24, and 12q13 loci are shown with the index SNP and additional SNPs within 500 Kbp in each direction, in the meta-analysis of SCAD GWAS discovery and replication groups. The association test used was the same as FIG. 2A. Similarly, the chromosome 21q22.11 locus region identified in the GWAS meta-analysis of female subjects is shown. LD r2 and recombination rate information were estimated based upon 1000G EUR population.

FIGS. 3A-E. Arterial expression of genes implicated by the SCAD GWAS meta-analysis. FIG. 3A, Colocalization analysis results in which the lead SNP identified matched the queried transcript are shown for each locus identified. The locus-compare scatter plot compares linear regression eQTL (n=913) and GWAS results (n=8,903), using the same method as FIG. 2A, in the gene region, which indicates whether the GWAS top locus is also the leading SNP in the eQTL result, supporting the conclusion that both traits are associated and share a single causal variant. P values are two-sided and unadjusted for multiple testing. The gene prioritized in each locus is shown on the y-axis and corresponding figure label. FIG. 3B, Boxplots of transcript expression levels by sex (n=913, which includes 593 males and 320 females) are displayed for each gene prioritized by the colocalization analysis. Transcript expression levels are measured in Transcripts Per Million (TPM). Gene TPMs were downloaded from the GTEx portal (v7) and subset to include only values from coronary artery tissue. The median of each gene is represented as the center horizontal line within each box, colored light purple for females and light blue for males. The top boundary of the box for each gene represents the 75th percentile of associated TPM values and the bottom boundary of the box represents the 25th percentile. The end point of the whiskers extending from each box mark the minimum and maximum TPM values respectively. FIG. 3C, Expression QTL results in GTEx arterial tissues. Violin plots depict normalized expression by allele. Two-sided values of linear regression P values listed represent calculated association between genotypes of each listed SNP and the corresponding eGene of interests referenced in FIGS. 3A and 3B. FIG. 3D, Arterial ADAMTSL4 immunostains of normal human coronary artery showing staining (brown) in the arterial media (20×), with nuclei stained blue. Inset bar=100 μm. FIG. 3E, In situ hybridization of normal human coronary artery with ADAMTSL4 mRNA (red) detected in the arterial media and with alpha actin (green) with nuclear DAPI staining (blue) co-localization to smooth muscle cells (40×, inset bar=50 μm), and magnified inset (inset bar=10 μm).

FIGS. 4A-B. PRS_{SCAD} and prevalent SCAD and MI events in a FMD cohort and the UK Biobank. FIG. 4A, Histogram of the weighted PRS_{SCAD} based on FMD cohort (N=412) with overlaying the corresponding data points in each interval. FIG. 4B, The OR's and corresponding 95% CI are shown are shown for the association of PRS_{SCAD} with SCAD-MI, all MI, or CAD/MI, in the FMD cohort (n=412, which includes 28 SCAD cases), UK Biobank (n=373,056, which includes 15,476 CAD/MI cases), and MVP (n=294,465, which includes 95,347 CAD cases; n=314,434, which includes 14,802 MI cases). The logistic regression Wald statistic of two-sided P values are displayed. All models are adjusted by age, sex, and PCs. P values are unadjusted for multiple testing.

FIGS. 5A-B. PheWAS phenotypic associations in the UK Biobank. FIG. 5A, PheWAS of the PRS_{SCAD} versus self-reported non-cancer medical illnesses in the UK Biobank (n=373,015). The different color dots represent the association results (logistic regression two-sided test P-values in minus log base ten scale) of the PRS_{SCAD} for non-cancer illness categories. Two-sided P values were considered significant when below a Bonferroni-adjusted threshold (0.05/2,356≈2.12×10⁻⁵). FIG. 5B, Sex-specific ORs in blue circles for males (n=171,082) or purple circles for females (n=201,974), are shown in the center of the corresponding 95% confidence interval (CI) from the PRS_{SCAD} analysis for migraine headache and MI, with two-sided values of P values displayed. Events in the legend denote the number of migraine cases or MI cases among the female or male groups, with N shown for the total sample size for each group. The models were adjusted for age at enrollment, genetic sex, genotyping array and batch, and the first four PCs. The logistic regression Wald statistic of two-sided P values are displayed.

FIG. 6. Illustration of SCAD.

FIGS. 7A-B. Ancestry estimation of SCAD samples.

FIG. 8. Females-only SCAD GWAS meta-analysis.

FIGS. 9A-C. GTEx tissue expression data for eQTL-associated genes.

FIGS. 10A-B. GTEx tissue expression data for the chromosome 21q22.11 locus.

FIG. 11. Phenome-wide association study (PheWAS) of UKB data.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a biomarker” is a reference to one or more biomarkers and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including human and non-human animals (e.g., dogs, cats, cows, horses, sheep, poultry, fish, crustaceans, etc.). As used herein, the term “patient” typically refers to a subject that is being treated for a disease or condition.

As used herein, the term “preventing” refers to prophylactic steps taken to reduce the likelihood of a subject (e.g., an at-risk subject, a subject suffering from acute internal tissue injury) from contracting or suffering from a particular disease, disorder, or condition. The likelihood of the disease, disorder, or condition occurring in the subject need not be reduced to zero for the preventing to occur; rather, if the steps reduce the risk of a disease, disorder or condition across a population, then the steps prevent the disease, disorder, or condition within the scope and meaning herein.

As used herein, the terms “treatment,” “treating,” and the like refer to obtaining a desired pharmacologic and/or physiologic effect against a particular disease, disorder, or condition. Preferably, the effect is therapeutic, i.e., the effect partially or completely cures the disease and/or adverse symptom attributable to the disease.

The terms “biological sample,” “sample,” and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), mucosal biopsy tissue and brushed cells, sputum, tears, mucus, nasal washes, nasal aspirate, breath, urine, semen, saliva, peritoneal washings, ascites, cystic fluid, meningeal fluid, amniotic fluid, glandular fluid, lymph fluid, nipple aspirate, bronchial aspirate (e.g., bronchoalveolar lavage), bronchial brushing, synovial fluid, joint aspirate, organ secretions, cells, a cellular extract, and cerebrospinal fluid. This also includes experimentally separated fractions of all of the foregoing. For example, a blood sample can be fractionated into serum, plasma, or into fractions containing particular types of blood cells, such as red blood cells or white blood cells (leukocytes). In some embodiments, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas, and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual. It will be appreciated that obtaining a biological sample from a subject may comprise extracting the biological sample directly from the subject or receiving the biological sample from a third party.

As used herein, the term “biomarker” refers to a measurable substance, the detection of which indicates a particular disease/condition or risk of acquiring/having a particular disease/condition. A “biomarker” may indicate a change in expression or state of the measurable substance that correlates with the prognosis of a disease. A “biomarker” may be a protein or peptide, a nucleic acid, or a small molecule. A “biomarker” may be measured in a bodily fluid such as plasma, and/or in a tissue (e.g., mammary tissue). In the context of the method described herein, a “biomarker” can be a single nucleotide polymorphism that is detected in a sample form a subject.

As used herein, the term “SNP” or “single nucleotide polymorphism” refers to a genetic variation between individuals; e.g., a single nitrogenous base position in the DNA of organisms that is variable. As used herein, “SNPs” is the plural of SNP. Of course, when one refers to DNA herein, such reference may include derivatives of the DNA such as amplicons, RNA transcripts thereof, etc. A “polymorphism” is a locus that is variable; that is, within a population, the nucleotide sequence at a polymorphism has more than one version or allele. One example of a polymorphism is a “single nucleotide polymorphism”, which is a polymorphism at a single nucleotide position in a genome (the nucleotide at the specified position varies between individuals or populations).

The term “allele” refers to one of two or more different nucleotide sequences that occur or are encoded at a specific locus, or two or more different polypeptide sequences encoded by such a locus. For example, a first allele can occur on one chromosome, while a second allele occurs on a second homologous chromosome, e.g., as occurs for different chromosomes of a heterozygous individual, or between different homozygous or heterozygous individuals in a population. An allele “positively” correlates with a trait when it is linked to it and when presence of the allele is an indicator that the trait or trait form will occur in an individual comprising the allele. An allele inversely correlates with a trait when it is linked to it and when presence of the allele is an indicator that a trait or trait form will not occur in an individual comprising the allele.

A marker polymorphism or allele is “correlated” or “associated” with a specified phenotype (e.g. SCAD, MI, migraine, etc.) when it can be statistically linked (positively or inversely) to the phenotype. That is, the specified polymorphism occurs more commonly in a case population (e.g., subjects suffering from FMD that also suffer from SCAD) than in a control population (e.g., subjects suffering from FMD that do not suffer from SCAD). This correlation is often inferred as being causal in nature, but it need not be—simple genetic linkage to (association with) a locus for a trait that underlies the phenotype is sufficient for correlation/association to occur.

The “polygenic risk score” is used to define an individuals' risk of developing a disease or condition, based on a multiple biomarkers, each of which might have modest individual effect sizes contribute to the disease or condition, but in aggregate have significant predicting value. In the present case, the polygenic risk score is used to predict the likelihood that a patient will develop SCAD, MI, or migraine using single nucleotide polymorphisms (SNPs) associated with the phenotype. For example, the odds ratio (OR) from every variant used in the calculation is used to calculate the polygenic risk score. In some embodiments, the Odds Ratio for each variant present in a subject is multiplied by the number of reference alleles (0, 1 or 2) carried by the individual. In some embodiments, the resulting additive score is standardized to the same measure in population controls by the same measurement amongst population controls, resulting in the final polygenic risk score. Other methods of manipulating the odds ratios, presence/absence of the biomarkers, normalizing/standardizing the risk score, including controls, etc. are within the scope herein.

“Predetermined cutoff,” “cutoff,” “predetermined level,” and “reference level” as used herein refer to an assay cutoff value that is used to assess diagnostic, prognostic, or therapeutic efficacy results by comparing the assay results against the predetermined cutoff/level, where the predetermined cutoff/level already has been linked or associated with various clinical parameters. It is well-known that cutoff values may vary depending on the nature of the test, condition, etc. It further is well within the ordinary skill of one in the art to adapt the disclosure herein for tests, risk scores, and/or specific cutoff values based on the description provided by this disclosure. Whereas the precise value of the predetermined cutoff/level may vary between assays, the correlations as described herein should be generally applicable.

“Risk assessment,” “risk classification,” “risk identification,” or “risk stratification” of subjects (e.g., patients) as used herein refers to the evaluation of factors including biomarkers, to predict the risk of occurrence of future events (e.g., SCAD, MI, etc.) including disease onset or disease progression, so that treatment decisions regarding the subject may be made on a more informed basis.

As used herein, the terms “prognosis,” “prognosticate,” and related terms refer to the description of the likely outcome of a particular condition, such as the likelihood of SCAD or MI in a subject.

### DETAILED DESCRIPTION

Provided herein are systems and methods for determining a subject's risk of spontaneous coronary artery dissection (SCAD) and myocardial infarction (MI) and systems and methods of using SCAD and/or MI risk for treatment thereof.

Spontaneous coronary artery dissection (SCAD) is a non-atherosclerotic cause of myocardial infarction (MI), typically in young women. Experiments conducted during development of embodiments herein used a genome-wide association study of SCAD (N_{cases}=270/N_{controls}=5,263) and identified and replicated an association of rs12740679 at chromosome 1q21.2 (P_{discovery+replication}=2.19×10⁻¹², OR=1.8) influencing ADAMTSL4 expression. Meta-analysis of discovery and replication samples identified associations with P<5×10⁻⁸ at chromosome 6p24.1 in PHACTR1, chromosome 12q13.3 in LRP1, and in females-only, at chromosome 21q22.1 near LINC00310. A polygenic risk score for SCAD was associated with (1) higher risk of SCAD in individuals with fibromuscular dysplasia (P=0.021, OR=1.82[95% CI:1.09-3.02]) and (2) lower risk of atherosclerotic coronary artery disease and MI in the UK Biobank (P=1.28×10⁻¹⁷, HR=0.91[95% CI:0.89-0.93], for MI) and Million Veteran Program (P=9.33×10⁻³⁶, OR=0.95[95% CI:0.94-0.96], for CAD; P=3.35×10⁻⁶, OR=0.96[95% CI:0.95-0.98] for MI). The experiments conducted during development of embodiments herein demonstrate that SCAD-related MI and atherosclerotic MI exist at opposite ends of a genetic risk spectrum, inciting MI with disparate underlying vascular biology.

The most common arteriopathy reported to co-occur with SCAD is fibromuscular dysplasia (FMD) (Refs. 2, 4-9; incorporated by reference in their entireties). Arterial dissections were reported in ˜26% of individuals with FMD (Ref 10; incorporated by reference in its entirety), including SCAD in 2.7% of patients in the updated US FMD registry. FMD is familial in some cases, with autosomal dominant inheritance pattern and incomplete penetrance¹¹⁻¹⁴. Familial studies of SCAD inheritance are lacking, although familial clustering has been observed (Refs. 15-16; incorporated by reference in their entireties). A common variant on chromosome 6p24.1 in the PHACTR1 gene, rs9349379-A (minor allele frequency ˜0.4), has been associated with both FMD and SCAD (OR_{FMD}=1.4, OR_{SCAD}=1.7) (Refs. 14, 17; incorporated by reference in their entireties). Notably, the same allele has also been associated with cervical artery dissection and migraine (Refs 18-21; incorporated by reference in their entireties), suggesting a common underlying genetic architecture. Less commonly observed in patients with SCAD are systemic inflammatory diseases in 5-12% (e.g. systemic lupus erythematosus, Crohn's disease) (refs. 1-2; incorporated by reference in their entireties) and monogenic vascular connective tissue diagnoses in fewer than 5% of cases (e.g. Marfan syndrome due to FBN1 pathogenic variation, or vascular Ehlers-Danlos syndrome due to COL3A1 pathogenic variation) (Refs 22-23; incorporated by reference in their entireties). The pathophysiology of SCAD may also be linked to female reproductive hormonal exposure, supported by the observation that 90% of SCAD cases occur in women, especially those who are young or middle aged (Refs 1-2, 24; incorporated by reference in their entireties).

Using a genome-wide association study (GWAS) approach, experiments were conducted during development of embodiments herein to identify multiple risk loci for SCAD and extend the relevance of the GWAS findings to an association with SCAD risk in a cohort of 412 individuals with FMD, as well as MI risk in more general populations through analysis of the UK Biobank (UKB) and Million Veteran Program (MVP).

An association of rs12740679 at the chromosome 1q21.2 locus was identified, implicating the extracellular matrix protein-encoding gene ADAMTSL4. Experiments also replicated a previously reported association of an intronic variant in the PHACTR1 gene, at chromosome 6p24.1, and identified associations of non-coding variants in the LRP1 gene at chromosome 12q13.3 and near MRP6/KCNE3 at chromosome 21q22.11. To explore pleiotropy and biologic underpinnings of SCAD and related arterial diseases, a genetic risk score, based upon the SCAD GWAS findings, was developed and tested. These analyses demonstrated association with SCAD occurrence in an independent FMD cohort analysis, as well as opposing risks of atherosclerotic-related MI and SCAD-related MI, in two large cohort studies. Further, the score was concordantly associated with increased risk of migraine headache and tinnitus, a clinical feature of FMD. Notably, the association of the SCAD risk score indicates sex-dimorphic and opposing influences on vascular biology, whereby one end of the spectrum of risk leads to arterial fragility and predisposition to arterial dissection with resulting MI, predominantly in women, and the other end of the spectrum of risk contributes to susceptibility to coronary atherosclerotic MI, a disease that affects both sexes but occurs more often in men.

Until recently, SCAD was rarely diagnosed, and little was known about the relevant vascular biology. Increased recognition of the disease, particularly in young women, and modern coronary angiographic methods have led to improved diagnoses, such that SCAD is now recognized as an important cause of MI in women less than age 50 years (Ref 1;

incorporated by reference in its entirety). Due to the female preponderance and peripartum occurrence in a small subset (<5%) of cases, hormonal factors have been implicated, but with little mechanistic data to support a specific role. SCAD and mFMD have overlapping phenotypes, both occurring predominantly in women (9:1 ratio of women to men with both diagnoses), and approximately 61% of our SCAD discovery cohort had mFMD. Both SCAD and FMD typically occur in individuals without a high burden of traditional risk factors for atherosclerosis, such as hypertension, hyperlipidemia, smoking, or diabetes. While luminal stenosis of the coronary arteries is not commonly observed in FMD, coronary arterial wall abnormalities have been documented (Ref 34; incorporated by reference in its entirety). FMD is currently understood as a likely genetically heterogenous condition with both sporadic and familial forms, with at least a partially complex genetic basis. While SCAD may occur in individuals with monogenic conditions such as Marfan Syndrome (due to FBN1 pathogenic variants), Loeys-Dietz syndrome, (due to TGFBR1/2 and other TGF-β pathway gene variants), vascular Ehlers-Danlos syndrome (due to COL3A1 variants), this is uncommon (<5%); no such molecular diagnoses have been defined for FMD.

The chromosome 1q21.3 locus regulates the arterial expression of the gene prioritized by colocalization analysis in this region, ADAMTSL4. ADAMTSL4 is a member of the ADAMTS (a disintegrin and metalloproteinase with thrombospondin motifs)-like gene family, and it encodes an extracellular matrix protein that binds to fibrillin-1 to promote the formation of microfibrils in the matrix (Ref 35; incorporated by reference in its entirety). ADAMTSL4 pathogenic variants underlie an autosomal recessive form of ectopia lentis (Refs. 36-38; incorporated by reference in their entireties), and fibrillin-1 gene variants may cause autosomal dominant ectopia lentis and Marfan Syndrome (Ref 39; incorporated by reference in its entirety). Histopathologic data localizing ADAMTSL4 protein and mRNA expression to the medial layer of the arterial wall, and medial vascular smooth muscle cells specifically, is consistent with the arterial media as the site of dissection and intramural hemorrhage (ref 5; incorporated by reference in its entirety). That women demonstrate higher basal expression of ADAMTSL4, and the allele conferring risk for SCAD is associated with lower expression of this gene, implicates a relative deficiency of ADAMTSL4 involved in a mechanism of promoting arterial fragility, as has been observed in disorders of fibrillin-1 deficiency (Ref 40; incorporated by reference in its entirety).

The chromosome 6p24 locus (rs9349379-A) associated with FMD (Ref 14; incorporated by reference in its entirety) and SCAD (Ref 17; incorporated by reference in its entirety) is located at an enhancer in aortic tissue. Mechanisms have been suggested for regulation of phosphatase and actin regulator 1 (PHACTRJ) (Ref 42; incorporated by reference in its entirety) and neighboring endothelin-1 (EDN1) gene transcription (Ref 43; incorporated by reference in its entirety). Low density lipoprotein receptor related protein (LRP1) encodes a cell membrane associated protein that interacts with a number of secreted proteins and cell surface molecules to mediate their endocytosis or the activation of signaling pathways. LRP1 GWAS-implicated genetic variants have been associated with migraine headache (Ref 19; incorporated by reference in its entirety) and abdominal aortic aneurysm (Ref 44; incorporated by reference in its entirety). Disruption of Lrp1 in vascular smooth muscle cells in mice leads to loss of vascular wall integrity and increased susceptibility to atherosclerosis (Ref 45; incorporated by reference in its entirety). The chromosome 21q22.11 locus prioritized genes include multidrug resistance protein-6 (MRP6), also known as ATP-binding cassette subfamily C, member 6, or ABCC6, which encodes for a cellular transporter, and pathogenic variants inherited in a recessive pattern have been described to cause pseudoxanthoma elasticum, a connective tissue disorder with characteristic arterial dysplasia characterized by calcification in elastic tissues.

Experiments conducted during development of embodiments herein demonstrate that SCAD, migraine headache, and FMD at least partly share a common genetic basis. Notably, the same SCAD-associated PHACTR1 locus and LRP1 locus alleles have been associated with cervical artery dissection (Ref 18; incorporated by reference in its entirety), which has been documented in 16.4% of individuals with FMD (Ref 10; incorporated by reference in its entirety), and migraine headache, which occurs in ˜36% of individuals with SCAD (Ref 8; incorporated by reference in its entirety) and ˜50% with FMD (Ref 10; incorporated by reference in its entirety). Despite the observed genetic pleiotropy of these loci, and that a substantial proportion of individuals with SCAD are also diagnosed with multifocal FMD, SCAD has been documented in only 2.7% of individuals with FMD (Ref 10; incorporated by reference in its entirety). As such, there is currently an unmet clinical need for risk stratification of individuals with FMD, to identify the subset at risk for SCAD-MI. By applying the PRS_{SCAD}, in a cohort of individuals with FMD, an association of the PRS_{SCAD} with SCAD events was identified. Clinical implications of identifying an individual with FMD who is at elevated SCAD risk include: 1) the consideration of antiplatelet therapy to prevent thrombotic complications in the event of a dissection, 2) a need for especially close blood pressure monitoring and control, 3) consideration of pregnancy risk (e.g., administering a contraceptive), 4) specific behavioral recommendations to reduce arterial strain (e.g., avoidance of isometric exercises), 5) avoidance of certain medications, such as triptans that have vasoactive properties and are commonly used to treat migraine headache and fluoroquinolone antibiotics which increase the risk of aortic dissection (Ref 46; incorporated by reference in its entirety), and 6) whether pharmacologic therapy may provide benefit for primary SCAD prevention (e.g., beta-blockers, to reduce arterial shear stress) (Ref 47; incorporated by reference in its entirety), or may cause harm.

Experiments conducted during development of embodiments herein demonstrate an inverse relationship of the PRS_{SCAD} with MI caused by more common atherothrombotic etiologies. The PRS_{SCAD} was protective against MI events. Lower PRS_{CAD} is associated with an increased risk of migraine headache, and conversely that migraine-associated genetic variants associate with reduced risk of CAD.

The disclosure provides a method comprising: (a) obtaining a biological sample from a subject (e.g., a subject suffering from FMD); and (b) assaying the sample for one or more biomarkers described herein (e.g., rs11207415, rs12740679, rs78377252, rs9349379, rs78349783, rs11172113, and rs28451064). As disclosed herein, the biological sample may be any biological material obtained or otherwise derived from an organism (e.g., a human). The biological sample may comprise, for example, saliva, blood, or a processed blood product. In some embodiments, obtaining a biological sample from a subject comprises extracting the biological sample directly from the subject or receiving the biological sample from a third party. In other embodiments, a biological sample may be extracted directly from a subject and sent to a third party for analysis.

In some embodiments, methods herein comprise detecting on or more of the biomarkers of Table 3 in a sample from a subject. In some embodiments, methods comprise detecting one or more (e.g., 1, 2, 3, 4, 5, 6, 7) of the biomarkers of Table 4 (e.g., rs11207415, rs12740679, rs78377252, rs9349379, rs78349783, rs11172113, and rs28451064) in a sample from a subject.

In some embodiments, methods herein comprise calculating a risk score (e.g., risk of atherosclerotic coronary artery disease and/or atherosclerotic-related MI, risk of SCAD and/or SCAD-related MI, etc.) based on the presence/absence of a combination of the biomarkers herein. In some embodiments, biomarkers contribution to the risk score is weighted by a factor related to the degree of correlation to a particular condition (e.g., SCAD, atherosclerotic-related MI, migraine, etc.). In some embodiments, the biomarkers are weighted according to their effect estimate, odds ratio, or any other suitable measure of correlation. In some embodiments, a polygenic risk score is calculated.

Exemplary methods for detecting the presence or absence of a biomarker include, but are not limited to, polymerase chain reaction (PCR)-based technologies including, for example, reverse transcription PCR (RT-PCR) and quantitative or real-time RT-PCR (RT-qPCR). Other methods include microarray analysis, RNA sequencing (e.g., next-generation sequencing (NGS)), in situ hybridization, and Northern blot.

In some embodiments, nucleic acid (e.g., DNA or RNA) may be isolated, purified, and/or amplified from the biological sample prior to assaying the biological sample. Commercially available kits and systems for isolating and purifying nucleic acid (e.g., DNA or RNA) may be used in connection with the disclosure.

In some embodiments, primers, probes, or other reagents for detecting the biomarkers herein are provided. The polymorphisms, corresponding marker probes, amplicons or primers described herein can be embodied in any system herein, either in the form of physical nucleic acids, or in the form of system instructions that include sequence information for the nucleic acids. For example, the system can include primers or amplicons corresponding to (or that amplify a portion of) a gene or polymorphism described herein. As in the methods herein, the set of marker probes or primers optionally detects a plurality of polymorphisms. Thus, for example, the set of marker probes or primers detects at least one polymorphism in each of these polymorphisms or genes, or any other polymorphism, gene or locus defined herein. Any such probe or primer can include a nucleotide sequence of any such polymorphism or gene, or a complementary nucleic acid thereof, or a transcribed product thereof (e.g., a nRNA or mRNA form produced from a genomic sequence, e.g., by transcription or splicing).

In some embodiments, the risk score is compared to a threshold level and the subject is diagnosed as being at elevated risk or reduced risk of condition based thereon (e.g., elevated risk of SCAD, reduced risk of MI, etc.). The terms “threshold level” and “reference level” may be used interchangeably herein to refer to an assay value that is used to assess diagnostic, prognostic, or therapeutic efficacy and that has been linked or is associated herein with various clinical parameters. It is well-known that threshold levels may vary depending on the nature of the assay and that assays can be compared and standardized.

Embodiments involve detection and analysis of multiple genetic variants (e.g. SNPs) which are used to calculate a polygenic risk score suitable for identifying individuals at a greater or lesser risk of developing a condition (e.g., SCAD, atherosclerotic-related MI, migraine, etc.). Detection methods for detecting relevant alleles include a variety of methods well known in the art, e.g., gene amplification technologies. For example, detection can include amplifying the polymorphism or a sequence associated therewith and detecting the resulting amplicon. This can include admixing an amplification primer or amplification primer pair with a nucleic acid template isolated from the organism or biological sample (e.g., comprising the SNP or other polymorphism), where the primer or primer pair is complementary or partially complementary to at least a portion of the target gene, or to a sequence proximal thereto. Amplification can be performed by DNA polymerization reaction (such as PCR, RT-PCR) comprising a polymerase and the template nucleic acid to generate the amplicon. The amplicon is detected by any available detection method, e.g., sequencing (e.g., next generation sequencing), hybridizing the amplicon to an array (or affixing the amplicon to an array and hybridizing probes to it), digesting the amplicon with a restriction enzyme (e.g., RFLP), real-time PCR analysis, single nucleotide extension, allele-specific hybridization, or the like. Genotyping can also be performed by other known techniques, such as using primer mass extension and MALDI-TOF mass spectrum (MS) analysis, such as the MassEXTEND methodology of Sequenom, San Diego, Calif. In certain embodiments, primers for amplification are located on a chip. Amplification can include performing a polymerase chain reaction (PCR), reverse transcriptase PCR (RT-PCR), or ligase chain reaction (LCR) using nucleic acid isolated from the organism or biological sample as a template in the PCR, RT-PCR, or LCR. In certain embodiments, the method further comprises cleaving the amplified nucleic acid. Other methods for detecting the biomarkers herein are understood in the field and applicable to embodiments herein.

In some embodiments, one or more additional steps are taken upon identifying a subject as having an elevated risk of SCAD. In some embodiments, methods further comprise a subsequent step of administering a treatment (e.g., therapeutic), such as aspirin, an antiplatelet therapy, a beta blocker, a contraceptive etc. In some embodiments, methods further comprise cessation or avoidance of a treatment or activity that increases the risk of SCAD, for example, cessation of treatment with triptan medications, avoiding activities that increase arterial strain or spikes in blood pressure. In some embodiments, methods further comprise additional monitoring, such as monitoring blood pressure, monitoring cardiac biomarkers, etc. In some embodiments, methods further comprise a subsequent step of screening said subject for comorbidities. In some embodiments, methods further comprise generating a report indicating the presence/absence of the biomarkers tested, a risk score generated, an elevated or reduced risk (e.g., of SCAD, of MI, of migraine, etc.), and/or steps to be taken.

In some embodiments, a subject is administered an antiplatelet therapy based on the results of testing performed according to the methods described herein. Exemplary antiplatelet therapies include aspirin, clopidogrel, dipyridamole, etc.

In some embodiments, a subject is administered a beta blocker (beta-adrenergic blocking agent) based on the results of testing performed according to the methods described herein. Exemplary beta blockers include acebutolol, atenolol, betaxolol, betaxolol, bisoprolol fumarate, carteolol, carvedilol, esmolol, labetalol, metoprolol, nadolol, nebivolol, penbutolol, pindolol, propranolol, sotalol, and timolol.

In some embodiments, method herein comprise counseling a subject at increased risk of SCAD about the SCAD-related risks of pregnancy and/or the benefits of contraception, and/or administering a contraceptive (e.g., drug, device, etc.) to a subject at increased risk of SCAD. Exemplary method of contraception include long-acting reversible contraception, such as the implant or intra uterine device (IUD), hormonal contraception (e.g., oral contraception, injection, etc.), barrier methods, emergency contraception, etc.

In some embodiments, a subject determined to be at elevated risk of SCAD is instructed to cease the use of triptan drugs (e.g., for the treatment of migraine). Exemplary triptan drugs include almotriptan, eletriptan, frovatriptan, naratriptan, rizatriptan, sumatriptan, and zolmitriptan.

In some embodiments, methods and systems are provided for assessing a risk of an individual developing a condition (e.g., SCAD, atherosclerotic-related MI, migraines, etc.). The method includes determining, in a biological sample from a human subject (e.g., a subject suffering from FMD), the presence or absence of two or more risk alleles (e.g., biomarkers, at independent loci, etc.). A polygenic risk score for the human subject can then be calculated based upon the presence or absence of the risk alleles and their relative correlation to the condition (e.g., odds ratio). In some embodiments, a risk allele that was demonstrated in the experiments conducted during development of embodiments herein to be more highly correlated with the condition is weighted more heavily in calculating the risk score. In some embodiments, a higher risk score (e.g., polygenic risk score) indicates a higher risk for developing the condition (e.g., SCAD, SCAD-related MI, etc.). In some embodiments, a higher risk score (e.g., polygenic risk score) indicates a lower risk for developing the condition (e.g., atherosclerotic-related MI).

### EXPERIMENTAL

### Methods

### Clinical Samples

Experiments were conducted during development of embodiments herein to investigate the genetics of SCAD in the large prospective cohort of patients enrolled in the Canadian SCAD (CanSCAD) Study. The CanSCAD study included SCAD patients from the prospective Canadian SCAD Cohort Study and the Non-Atherosclerotic Coronary Artery Disease (NACAD) Study. Patients presenting with acute SCAD were prospectively enrolled from 22 sites throughout North America (20 sites in Canada and 2 in the United States). SCAD diagnosis was confirmed on coronary angiography by the UBC core laboratory research team, and categorized according to previously established Saw classification (Refs. 51-52; incorporated by reference in their entireties). Type 1 SCAD depicts contrast dye staining of arterial wall with multiple radiolucent lumen, with or without dye hang-up or slow contrast clearing from the lumen. Type 2 SCAD depicts diffuse and smooth narrowing that varies in severity; Type 2A describes presence of normal arterial segments proximal and distal to dissection; Type 2B describes dissection that extends to distal tip of the artery. Type 3 SCAD depicts focal or tubular stenosis that appears similar to atherosclerosis. Intracoronary imaging with optical coherence tomography or intravascular ultrasound was performed at the discretion of the treating physicians to aid angiographic diagnosis. Detailed baseline demographics, targeted history for predisposing conditions and precipitating stressors, and laboratory screening for predisposing conditions were performed. Screening for FMD was recommended for all SCAD patients, and mFMD was defined according to consensus guidelines (Ref 24; incorporated by reference in its entirety). Patients were prospectively followed post-discharge at 1, 6, and 12 months, and annually thereafter for 3 years for cardiovascular (CV) events.

In the CanSCAD Genetics Substudy, site-specific research ethics board approvals for the study and individual patient consents were obtained. Genetic studies were performed on CanSCAD patients who provided informed consent. Collection of DNA was obtained through blood or saliva self-collection kit (Oragene-500 kit, DNAGenotek). DNA was extracted according to the manufacturer's instruction (DNAGenotek), and quantified using the Quant-iT PicoGreen assay (Life Technologies). DNA samples were normalized to a concentration of 50 ng/μl for genotyping. The processed DNA samples were batched and transferred to the University of Michigan for GWAS analysis.

Adult subjects with mFMD (N_{unrelated}=412) were enrolled in with IRB approval in the Cleveland Clinic FMD Biorepository. All participants provided informed consent and study activities were approved by the enrolling institution's IRBs. Each research participant contributed either a blood or saliva sample via standard K⁺ EDTA blood collection tubes or commercial saliva collection kits (Oragene, DNAGenotek). DNA was isolated according to commercial kit protocols (Nucleospin Tissue (TakaraBio)), extracted according to the prepIT-L2P extraction kit (DNAGenotek) and quantified using the Quant-iT PicoGreen dsDNA kit (ThermoFisher).

The Michigan Genomics Initiative (MGI) recruited participants while awaiting diagnostic, interventional, and surgical procedures. Participants provided a blood sample for genetic analysis and agreed to link their sample to their electronic health record and other sources of health information (Ref 54; incorporated by reference in its entirety). Analyses involved 13,756 individuals from MGI genotyped with the same version (v1.1) of the Illumina BeadArray genotyping platform as the SCAD and FMD cases at the University of Michigan DNA Sequencing Core Facility. ICD codes corresponding to diagnoses of arterial aneurysm, dissection and non-atherosclerotic dysplasia and stenosis were excluded, as well as connective tissue disorders.

The Cleveland Clinic GeneBank study is a sample repository generated from consecutive patients undergoing elective diagnostic coronary angiography or elective cardiac computed tomographic angiography with extensive clinical and laboratory characterization and longitudinal observation. Ethnicity was self-reported and information regarding demographics, medical history, and medication use was obtained by patient interviews and confirmed by chart reviews. All patients selected as controls were age and sex matched to the FMD cohort and had no evidence of coronary artery disease, defined as adjudicated diagnoses of stable or unstable angina, myocardial infarction (adjudicated definition based on defined electrocardiographic changes or elevated cardiac enzymes), angiographic evidence of 50% stenosis of one or more major epicardial vessel, and/or a history of known coronary artery disease (documented infarction, coronary disease, or history of revascularization). All patients provided written informed consent prior to being enrolled in GeneBank and the study was approved by the Institutional Review Board of the Cleveland Clinic.

The UK Biobank recruited adults of 40-69 years-of-age from across the United Kingdom (Ref 55; incorporated by reference in its entirety). Participants were assessed at enrolment via medical histories, physical exams, and biochemical measurements. Participant data is linked to hospital episode statistics.

The Million Veteran Program (MVP) (Ref 56; incorporated by reference in its entirety) recruited active users of the Veteran Health Administration (VA) of any age from more than 60 VA Medical Centers nationwide, with current enrollment at >825,000. Informed consent is obtained from all participants to provide blood for genomic analysis and access to their full electronic health record (EHR) data within the VA prior to and after enrollment. Imputed genetic information is available for up to 314,434 participants assigned to white-European ancestry using the HARE algorithm (Refs. 57-58; incorporated by reference in their entireties). Inpatient and outpatient International Classification of Diseases (ICD9/10) diagnostic and Current Procedural Terminology (CPT) codes were used to identify subjects with clinical CAD. An individual was classified as a case if he or she had ≥1 admission to a VA hospital with discharge diagnosis of acute myocardial infarction (AMI) OR≥1 procedure code for revascularization of the coronary arteries OR≥2 ICD codes for CAD (410 to 414) on ≥2 dates. Individuals with only 1 ICD code for CAD on 1 date and no discharge diagnoses for AMI or revascularization procedures were excluded from the analyses and remaining subjects were classified as controls. This algorithm identified up to 95,347 unrelated subjects with CAD and 199,118 unrelated controls with 19,969 subjects being excluded due to ambiguous CAD status. Subgroup analysis was performed involving the subset of cases with evidence of a hospitalization for MI. These cases were compared to all controls in association analysis (N=14,802 unrelated MI cases and N=299,632 unrelated controls).

### Genotyping on the Illumina Genotyping Chip and Variant Calling

Genotyping of SCAD, FMD, and MGI samples were conducted by the University of Michigan DNA Sequencing Core using the Illumina Infinium HTS Assay Protocol, a semi-custom Infinium CoreExome-24v1.1 BeadArray with 607,778 SNP markers (UM_HUNT_Biobank_v1-1_20006200_A), and the Illumina GenomeStudio v2011.1. This GWAS+exome chip platform includes standard genome-wide tagging SNPs (N-240,000), exomic variants (n-280,000) and custom content from previously published GWASs, additional exonic variants selected from sequencing studies, ancestry informative variants and Neanderthal variants. Data Analysis Software package with Genotyping Module v1.9.4 and Illumina GenomeStudio (version 2.0) were used to cluster and call genotypes. Sample filtering was performed to exclude samples with call rate <98%, estimated contamination >2.5% (BAF regress), chromosomal missingness greater than 5 times other chromosomes, and sex mismatch between genotype-inferred sex and reported gender. Variant filtering was performed to exclude probes that could not be perfectly mapped to the human genome assembly (Genome Reference Consortium Human genome build 37 and revised Cambridge Reference Sequence of the human mitochondrial DNA; BLAT); Hard Weinberg equilibrium deviations in European ancestry samples (P<0.00001); variant call rate <98%. Basic quality control (QC) filters including HWE P<0.000001, and variant missing call rate >2%, were implemented for each lab chip data and MGI chip data, before combining the two data sets.

All genotype data from SCAD and FMD cases were merged and then applied pre-HRC-imputation quality control using the HRC Imputation preparation and checking tool by the McCarthy Group [https://www.well.ox.ac.uk/˜wrayner/tools/] before merging them with MGI genotyped data, which applied the same pre-HRC-imputation QC. It compares each of our individual genotyped data with HRC reference, and corrects the variants strand-flip as well as aligns the allele codes with HRC reference alleles. It also removes A/T & G/C SNPs if MAF>0.4, SNPs with differing alleles, SNPs with >0.15 allele frequency difference, and SNPs not in reference panel. After this process, 351,487 polymorphic variants remained (chr1-23). The total genotyping rate was 0.99.

### Discovery Study SCAD Sample QC

277 cases with SCAD were collected by the University of British Columbia (UBC). Based on the genotyping result, one duplicate was excluded based on whole genome genotyping data identity-by-descent (IBD) analysis, two gender mismatched samples, and two that are further confirmed not SCAD cases. Genetic syndrome cases of SCAD (n=2) were identified and these individuals were removed from the analysis groups. All samples have missing call rate <1%. None of them fail QC in inbreeding coefficient check. In total, 270 SCAD samples were in the final GWAS study, which included 29 men and 241 (89.30%) women. Their average age was 53.3±9.7years. 13756 MGI control subjects were included in the initial database (54.8% women, average 53.1±16.4yrs). Two were removed due to IBD analysis that recognized them as duplicates. All samples passed gender and inbreeding coefficient check; and have missing call rate <1%. For the merged data, it was confirmed that none of the SCAD cases and MGI controls were duplicates or overlapped based on the IBD analysis. Duplicates were removed, but related samples were retained. Most of the sample QC were done by PLINK (Ref 59: incorporated by reference in its entirety).

### Replication Study SCAD Samples QC

165 cases with SCAD were collected for replication analysis. The same sample QC as discovery stage study was applied. One sample was r4emoved that is confirmed not SCAD case and one sample that has missing call rate >2%. No genetic syndrome cases were identified. 163 SCAD cases were used in the replication. The average age was 50.5±10.4yrs, which included 90% women. They were genotyped using the same GWAS array as the discovery cohort and imputed together with our discovery stage samples, but were only examined for the top variants of discovery result. The association was tested using SAIGE with the same study design, which were age, sex and ancestry (LASER/TRACE PCs) matched controls from the MGI samples, which were exclusively independent samples from previously used controls. It was confirmed that there were no overlapping samples between discovery stage and replication based on combining IBD analysis. The sample size for the SCAD replication analysis was 163 UBC SCAD cases and 3,207 MGI controls (1 to up to 21 ratio).

### Imputation to Haplotype Reference Consortium

After quality controls described above. Autosomal chromosome genotypes of the Haplotype Reference Consortium (HRC) were imputed using the Michigan Imputation Server on the 13,756 MGI and 433 UBC SCAD (discovery+replication) samples (Refs. 27, 60; incorporated by reference in their entireties). The parameters for imputation included: 1) Minimac4 method; 2) HRC r1.1 2016 reference panel; 3) Eagle v2.3 as phase output; 4) EUR as quality control population. Poorly imputed variants (R²<0.8) and rare variants (MAF<1%) were filtered. SNPs with potential frequency mismatches comparing with reference panel (markers with Chi-squared greater than 300) were excluded. There were 6,690,240 imputed variants (chromosomes 1-22) after the filter. The correlation r² between the ref allele frequency of the samples and the HRC reference panel was 0.999.

### Ancestry Estimation by Principal Components Analysis

TRACE in LASER (Locating Ancestry from Sequence Reads) software v3.0.0 were used to compute 5 principal components based on the genotype data to map the individual's genetic ancestry using world-wide HGDP samples as reference (Ref 61; incorporated by reference in its entirety). It applied principal components analysis (PCA) of the reference panel to construct K-dimensional reference ancestry space using K principal components. Two main clusters were observed in the SCAD while generating the PCA plots (PC1 to PC2). One cluster located in East Asia (N=25 for discovery samples, and N=34 for discovery+replication samples) and the other spread through Europe to Central South and West Asia (N=245 for discovery samples, and N=399 for discovery+replication samples). As a sensitivity analysis, the 25 East Asian samples were excluded to run the discovery stage association analysis.

### Matching of MGI Controls to SCAD Cases

In the case control matching design, to reduce the possible false-positives in our GWAS result, it was required that controls have the same gender, close birth years, and close ancestries. It was expected that every case could be matched to at least one control. A greedy approach was taken that searched from +/−5 years (5-year window) in age first, followed by +/−10 years instead, and so on to +/−30 years. Searching was stopped once there was at least one control selected. From the possible controls in the applicable sex and age category, the best ethnic match was selected for each case that had the smallest principal components distance (via the top 3 PCs given by TRACE program in LASER server (Refs. 62, 63; incorporated by reference in their entireties). To guarantee every case could match to at least one and the maximal number of controls, the entire procedure 21 times (this number is decided via testing back and forth) until all of the available controls were used that were filled in the selection criteria. In the final study, there were 270 SCAD cases (53.3±9.7yrs old, 89.3% female) and 5,263 matched controls (52.9±15.3yrs old, 88% female) that was used in discovery stage GWAS, and 163 SCAD cases (50.5±10.4yrs old, 90% female) and 3,207 matched controls (49.2±15.5yrs old, 89% female) in replication study (case control: 1 to up to 21 ratio).

### Genome-Wide Association Analysis Accounting for Imbalanced Case: Control Ratios

SAIGE (Ref 26; incorporated by reference in its entirety) was used, which introduces a scalable and accurate generalized mixed model association test that utilizes the saddle-point approximation to calibrate the distribution of score test statistics. SAIGE provides more accurate P values even when case-control ratios are extremely unbalanced, efficiently controlling and minimizing the type I error rates due to case-control imbalance and sample relatedness in large-scale genetic association studies. In the discovery stage and replication stages, association testing was performed for the SCAD status using SAIGE program for single genetic variants, with the first five principal components as covariates. 6,690,240 SNPs (after filter of imputation r²≥0.8 and MAF≥0.01) were tested in the discovery stage and the SNPs with P<5×10⁻⁸ were re-examined in replication stage using SAIGE. SNPs that retained P<0.05/(number of tested SNPs) in the replication analysis were reported. Manhattan plots and QQ plots were generated from genome wide SNPs. λ_{GC} was evaluated for population stratification.

### Meta-Analysis of SCAD Discovery and Replication GWAS Results

Individual genome-wide SAIGE association results of discovery stage and replication stage were then taken to meta-analysis. The METAL program (Ref 64; incorporated by reference in its entirety) was executed to combine P values across discovery and replication studies by taking into account each study weights effect size estimates using the inverse of the corresponding standard errors. Genomic control correction was applied to all input files of whole genome data. Manhattan plots and QQ plots were inspected as well. To visualize GWAs results for the top loci, gene locus plots (index SNP+/−500 Kb) for the top loci were generated using LocusZoom tool (Ref 65; incorporated by reference in its entirety). All SNPs with P<1.0×10⁻⁴ in the SCAD meta-analysis were reported.

### Identification of Independent SCAD-Associated Loci

From the all SAIGE association results and the meta-analysis result, the PLINK (Ref 59; incorporated by reference in its entirety) function “clump” was utilized for P<0.0001 variants by LD r²>0.2 in a window size of +/−500 Kb to obtain the independent loci. A further conditional test was conducted using SAIGE to test the independency of two loci whose distance was less than 1 Mb in the same chromosome after clump. Individual conditional results in discovery studies and replication studies by the top SNP are taken to meta-analysis by METAL. After conditioned by the top SNP, the strength of the significance of the tested SNPs was examined.

### FMD Sample QC and Analysis of SCAD-Associated Loci

Clinical replication sources were based on 412 adult FMD samples These samples underwent the same QC as we had for SCAD samples, which included excluding duplicates, samples with gender miss-match, and samples with missing call rate>1%. No samples failed QC due to inbreeding coefficient checks. IBD analysis was performed to confirm no duplicates or closely related samples (IBD PI-HAT<0.35) existed. The mean age was 53.3±10.9 years, and 97.8% of FMD cases were women. Samples were collected for clinical inspection for their association between the number of risk alleles in top SCAD SNPs and the status of dissection, aneurysm, and SCAD. These samples were genotyped using the same GWAS array as the discovery cohort and imputed together with our SCAD GWAS samples, but were only examined for the top variants identified by our SCAD analyses. Informed consent was obtained from all participants and approval was obtained from respective Institutional Review Boards.

### SCAD Heritability Estimation

A univariate linear mixed model (LMM) was fitted for estimating the proportion of variance explained (PVE) by typed genotypes (i.e. “SNP heritability”) using GEMMA, a software implementing the Genome-wide Efficient Mixed Model Association algorithm for genome-wide association studies (Ref 66; incorporated by reference in its entirety). The discovery SCAD GWAS whole imputation data was used for analysis using a univariate linear mixed model, and SNPs with MAF greater than 0.05 were included in the analysis. The individual-level data used in the analysis includes 5,577 samples and 5,020,100 SNPs. Restricted maximum likelihood estimate (REML) average information (AI) algorithm in GEMMA was used for estimating PVE.

### Transcript Expression Analysis of SCAD-Associated Loci in GTEx

Based on our SCAD-associated loci, the GTEx portal (Ref. 28; incorporated by reference in its entirety) was used to compare genes prioritized in the colocalization analysis in different tissues, and differences according to sex. Major eQTL associations (FDR≤0.05) were queried based on specific associated alleles, such as rs12740679 (G/C) and ADAMTSL4(ENSG00000143382.14), inr aorta, coronary artery, and tibial artery. Significant variant-gene associations from GTEx v7 was based on permutations with q-value<0.05. Sex differences were determined through DESeq analysis of GTEx RNA-Seq V7, and visualized through boxplots of TPM by sex. DESeq estimates variance-mean dependence in count data from high-throughput sequencing assays and tests for differential expression based on a model using the negative binomial distribution. Violin plots and corresponding P-values of the normalized transcript expression levels for carriers with zero risk alleles for SCAD, one risk allele, and two risk alleles in for each lead SNP were obtained through the GTEx eQTL Dashboard. Associations were calculated by linear regression, methods of which are listed in the reference for GTEx V7 (Ref 72: incorporated by reference in its entirety). For comparison, similar analyses were performed for additional top loci identified with P<5×10⁻⁸ in the GWAS meta-analyses.

### Colocalization Analysis to Prioritize Genes in the SCAD-Associated Loci

For the significant eQTLs identified by single variant GTEx eQTL querying for the SCAD associated SNPs, the colocalization analysis was performed as a follow-up confirmation, to test whether the leading variant of the GWAS and the eQTL signal is the same. A GWAS locus that colocalized with eQTL should be one of the primary and scalable candidate signals for follow-up functional and mechanism analyses. Two tools were utilized: coloc (Ref 67; incorporated by reference in its entirety) and locuscompareR (Ref 68; incorporated by reference in its entirety) in R program, both of which compare between eQTL result and GWAS result, taking into account LD information in the targeted gene region. The eQTL data-set was downloaded from GTEx Analysis V7 (dbGaP Accession phs000424.v7.p2), and the combining results across coronary, tibial and aorta arterial tissues were retrieved for each transcript. The meta-analysis result of SCAD GWAS analysis was compared with the eQTL result for each gene. (Approximate) Bayes Factor (ABF) colocalization analyses were adopted, which embedded the concept that association of each trait with SNPs in a region may be summarized by a vector of 0s and at most a single 1, with the 1 indicating the causal SNP (assuming a single causal SNP for each trait). The posterior probability of each possible structure can be calculated as well as the posterior probabilities that the traits share their structures. The function coloc.abf( ) in coloc was used to test the posterior probabilities for: (H0) neither trait has a genetic association in the region; (H1/H2) only one trait has a genetic association in the region; (H3) both traits are associated, but with different causal variants; (H4) both traits are associated and share a single causal variant. A posterior probability of (H4)≥75% suggests strong evidence of the eQTL-GWAS pair influencing both the expression and GWAS trait at a particular region. The locuscompareR package further helps to visualize the colocalization events, which generates a combined plot with two locus-zoom plots (eQTL and GWAS in the same gene region) and a locus-compare scatter plot (eQTL −log10(P) to GWAS −log10(P)). The figure indicates whether the GWAS top locus is also the leading SNP in the eQTL result (both traits are associated and share a single causal variant).

### Immunohistochemistry and RNA In Situ Hybridization to Assess Human Coronary Artery ADAMTSL4 Expression

Human coronary arteries were fixed and paraffin embedded. De-paraffinized sections were treated with target retrieval solution DAKO citrate pH 6.1 (Agilent, Santa Clara Calif.). Slides were labeled with anti-ADAMTSL4 (Sigma, St. Louis Mo.) on the DAKO Autostainer (Agilent, Carpinteria, Calif.) using ImmPRESS Rb (Vector labs, Burlingame, Calif.) and diaminobenzadine (DAB) as the chromogen. Slides were imaged on EVOS microscope (ThermoFisher, Waltham Mass.).

In situ hybridization for ADAMTSL4 mRNA was performed on a normal human coronary artery sample. 5-micron sections were obtained from the fixed and paraffin embedded human coronary artery, and slides were processed according to RNAscope Multiplex Fluorescent V2 assay manufacturer instructions (ACDbio, Newark Calif.). The probes used were as follows: C1-Hu TAGLN, C2-Hu ADAMTSL4, C3-HU ACTA2. Opal fluorophores (520, 570, 690) were used at 1:1000 dilution. (Akoya Biosciences, Marlborough, Mass.). Slides were imaged on Nikon Ti microscope using Element Software (Nikon, Melville N.Y.).

### Analyses of Pleiotropy

The top independent loci with false discovery rate q value <0.05 in the SCAD GWAS meta-analysis was selected after independent loci with LD pruning of r²>0.2, +/−500 Kb and demonstrating independence after the conditional analyses (N=7 loci), to search for derived PheWAS association (P<1.0×10⁻⁴) in the UKB database (/geneatlas.roslin.ed.ac.uk/phewas/). The FDR analysis was conducted based on meta-analysis result (P values) of 324,087 genome-wide LD-pruning SNPs that have MAF>1% and after clumping of r2>=0.2 at +/−500 Kb window with no P value filter, via R package qvalue (Ref 69; incorporated by reference in its entirety).

### Polygenic Risk Scores

To investigate whether the top SCAD loci are predictive for features in the FMD cases, the polygenic risk score (PRS) analysis was conducted by using the polygenic scores from 7 independent loci (false discovery rate q value <0.05 from 324,087 LD pruning genome-wide loci of SCAD GWAS meta-analysis and conditional test). These 7 SNPs comprised the PRS_{SCAD}. This study only focused on FMD cases. Logistic regression was conducted to test the association of the weighted PRS (aggregated number of risk alleles weighted by beta in our SCAD GWAS), and as a sensitivity analysis the unweighted PRS (sum of SCAD-associated risk alleles), or individual SNPs, with the binary status of sub-types in dissection, aneurysm, and FMD, including age and sex as covariates.

For the genotyping array analysis of DNA from UK Biobank participants, the PRS_{SCAD} was evaluated in 373,056 individuals of British white genetic ancestry. Weighted polygenic scores were calculated as a continuous variable using the equation: Σ(β₁*SNP₁)+ . . . +(β₇*SNP₇), where βₓ denotes the beta coefficient for the association of SNPₓ with risk of SCAD and SNPX denotes the number of SCAD-associated risk alleles (0, 1, or 2).

For the MVP cohort, the association between PRS_{SCAD} and CAD/MI status was tested using logistic regression adjusted for age (at the time of event in cases and at the time of last VA visit prior to August 2018 for controls), sex, and the first 10 ethnic-specific genetic principal components.

### PRS_{CAD} Evaluation in SCAD

Autosomal SNPs reported in the literature were tested as associated with CAD by GWAS-Catalog (Ref 32; incorporated by reference in its entirety) reported traits of coronary artery disease (CAD) and CAD itself (defined as MI, percutaneous transluminal coronary angioplasty, coronary artery bypass grafting, angina or chromic ischemic heart disease). For further PRS analysis, variants without risk allele or beta information reported and without data in our SCAD GWAS result were removed. All reported SNPs were LD-pruned based on r²>0.2 in +/−500 KB of index SNPs. If same variant was reported in multiple publications, the ones with strongest effect estimate (beta) reported were selected. There were totally 386 CAD associated SNPs selected for final PRS_{CAD} analysis. A polygenic risk score analysis was conducted using GTX tool package (www.rdocumentation.org/packages/gtx/versions/0.0.8) in R, which uses the summary statistics of our meta-analysis results of the SCAD discovery and replication GWAS results from SAIGE. The beta coefficients of the same loci were aligned to the same effect allele between published CAD association data and our SCAD GWAS data.

### Linkage Disequilibrium Assessment for Neighboring Signals in the Top-Ranked SCAD Loci

For the SNPs with P<1.0×10⁻⁸ in our top chr1q21.2-chr1q21.3 region, LD R-square was examined using the Phase 3 (Version 5) of 1000 Genomes Project CEU sub-population references by the LDlink program^{44,70}, which is a web-based tool to interrogate linkage disequilibrium in population groups. Low correlation corresponded to r<0.2.

### UK Biobank Cox-Regression Models for Risk of Myocardial Infarction

MI events were defined pre-enrollment by self-reported medical history and post-enrollment by hospital episode statistics using International Classification of Diseases, Version 10 diagnosis codes (121, 122, 123, or 124). Events were censored on the date of loss-to-follow-up, death, or if individuals remained event-free.

Time-to-event analyses were performed with the “survival” version 2.43-3 package for R version 3.5.1 using unadjusted and adjusted Cox-regression models with years of age as a timescale. Cox regression models were adjusted for genetic sex (when not stratified by genetic sex), genotyping array and batch, and the first 4 principal components of ancestry. Tests for interaction were assessed between genetic features and sex.

### UK Biobank Phenome-Wide Association Study

Odds ratios stratified by genetic sex were calculated for the association of the PRS_{SCAD} of 7 top ranked SNPs identified in the main SCAD GWAS meta-analysis that was described before with self-reported history of MI and migraine. Results were derived from logistic regression analyses adjusted for age at enrolment, genotyping array and batch, and first 4 principal components of genetic ancestry.

PHESANT software was used for R version 3.5.1 to perform a phenome-wide association study of weighted and continuous PRS_{SCAD} with 2,356 phenotypes related to self-reported history of cancers, non-cancer illnesses, operations, and medications assessed at study enrolment (https://github.com/MRCIEU/PHESANT) (Ref 71; incorporated by reference in its entirety). Analyses were performed using a logistic regression with the covariates of age at enrolment, genetic sex, genotyping array and batch, and first 4 principal components of genetic ancestry with standardization of weighted PRS_{SCAD} variable. Two-sided P values were considered significant when below a Bonferroni-adjusted threshold (0.05/2,356≈2.12×10⁻⁵).

### Results

### Phenotypic Characteristics of the SCAD Cohort

The SCAD discovery analysis was performed using samples from the Canadian SCAD Study (CanSCAD) (N_{cases}=272) (Ref 25; incorporated by reference in its entirety). Each participant's SCAD diagnosis was verified by coronary angiography and adjudicated by a core angiographic laboratory. Consistent with prior descriptions, 89% of the discovery sample was female, 2.6% of cases presented during the peripartum period, and the mean age of individuals presenting with SCAD was 53.2±9.7 years. Upon review of the medical record, genetics referrals, and genetic testing results, one individual was found to have Loeys-Dietz syndrome with a pathogenic variant in TGFBR2 (c.1591G>A, p.Ala531Thr), and one individual had a clinical diagnosis of Marfan Syndrome. FMD was identified in 60.9% of SCAD cases. The demographics and clinical characteristics of the discovery sample are summarized in Table 2.

### GWAS of SCAD

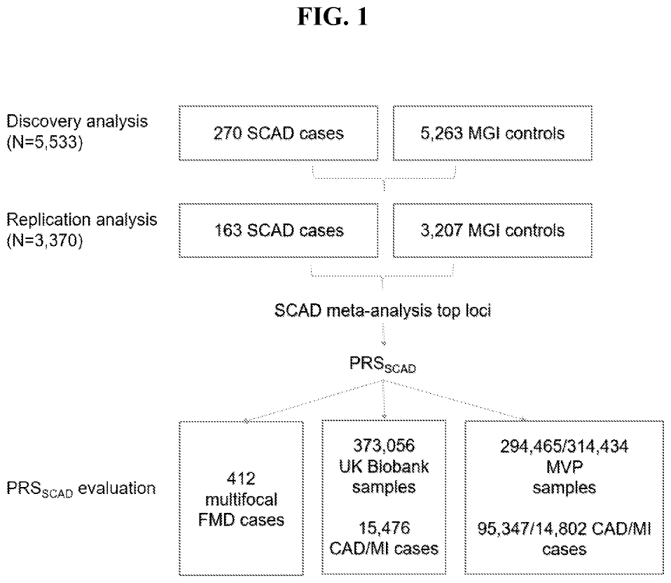

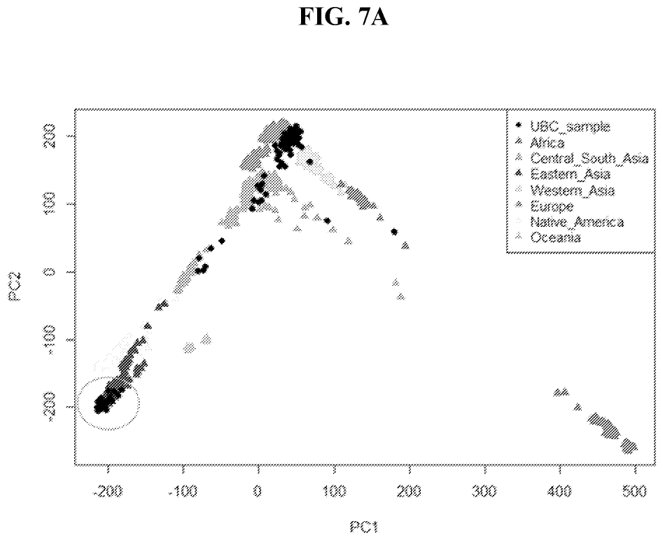

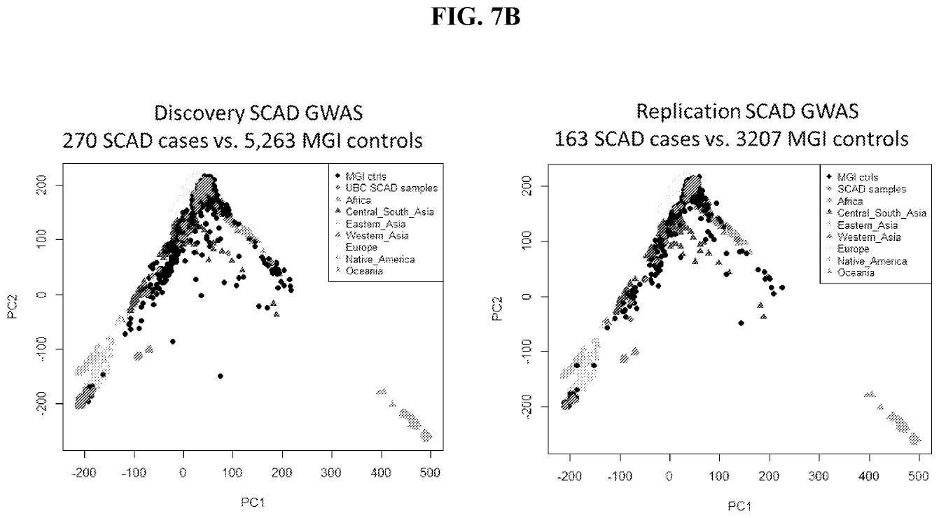

To discover genetic variants influencing the risk of SCAD, a GWAS of SCAD was undertaken, utilizing samples from the CanSCAD Study and control subjects without vascular disease from the Michigan Genomics Initiative (MGI) biorepository (FIG. 1). In order to maximally leverage the biorepository resource and analyze the largest sample size possible, the SAIGE method was employed to conduct association analyses accounting for imbalanced case:control ratios (Ref 26; incorporated by reference in its entirety). The discovery analysis was comprised of 270 successfully genotyped SCAD samples from individuals, after excluding two individuals with genetic syndrome diagnoses. Using MGI demographic data and principle components analysis, age, sex and ancestry-matched control subjects were identified in the MGI biorepository of >50,000 individuals who had provided consent for genetic study and access to electronic health records, and who did not have vascular disease or connective tissue disorder diagnoses. Ancestry estimation of our SCAD cases was performed by plotting PCs against world-wide Human Genome Diversity Project (HGDP) groups. The majority of discovery SCAD samples were predominantly European ancestry, and 9% were either mixed or predominantly East Asian ancestry (FIG. 7). Up to 21 age-, sex- and PC-matched controls were selected for each SCAD case (N_{controls}=5,263). Genotyping of both CanSCAD and MGI samples was successfully performed using the Illumina Human CoreExome BeadArray v1.1 genotyping array, with 607,778 genotyped variants. Genotype quality control and imputation to the Haplotype Reference Consortium (Ref 27; incorporated by reference in its entirety) reference panels were performed on case and control samples together, and 6,690,240 variants (imputation Rsq≥0.8 and MAF≥1%) were analyzed in 5,533 case and control samples in the discovery analysis.

SCAD GWAS identified rs12740679 at chromosome 1q21.2 to be significantly associated with SCAD (P=2.9×10⁻¹⁰, MAF=0.26, OR=1.97[95% CI:1.60-2.43]) (Table 1). A sensitivity analysis confirmed no significant ancestry-specific effect of including individuals of East Asian ancestry in the discovery analysis. GWAS results were compared after removing individuals of Asian ancestry to GWAS results after removal of a comparable number of individuals of non-Asian ancestry from the discovery GWAS, over 10 iterations. In all analyses, the chromosome 1q21.2 locus demonstrated P<5×10−8 and the distribution of results was not different between the removal of individuals of Asian ancestry as compared to the removal of individuals of non-Asian ancestry (permutation P=0.4) A replication analysis was conducted in similarly acquired samples from the CanSCAD study (N=163) of individuals enrolled from 2018 to 2019. The replication samples were genotyped on the same platform, matched to unique MGI control subjects by age, sex and PC-estimated ancestry that had not been previously included in the discovery GWAS (N_{controls}=3,207). Quality control and imputation to the HRC reference panel were performed on replication case and control samples together. The chromosome 1q21.2 locus was replicated in the independent analysis (OR=1.56[95% CI:1.21-2.02], P=7.32×10⁻⁴) (Table 1).

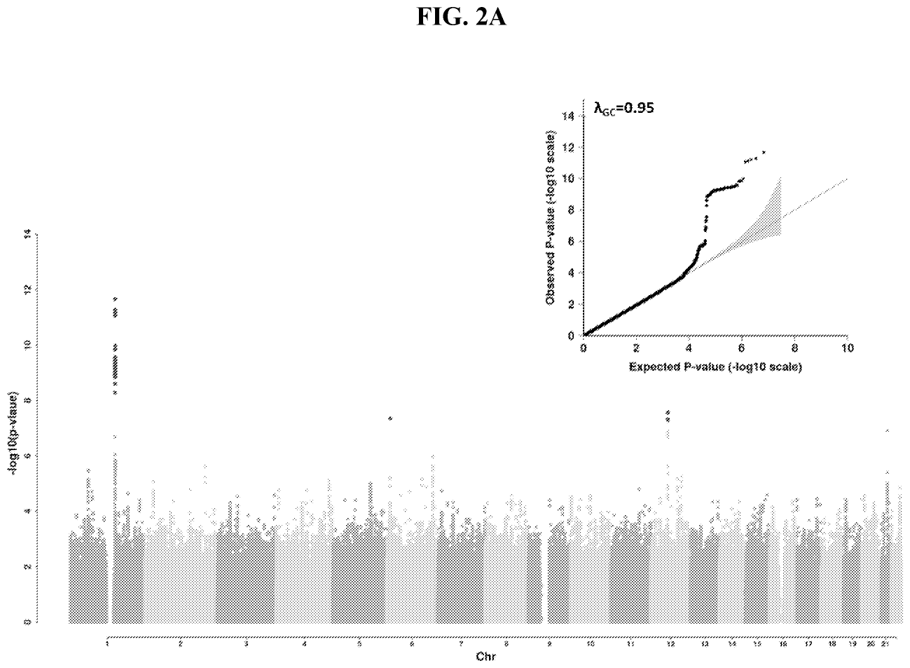

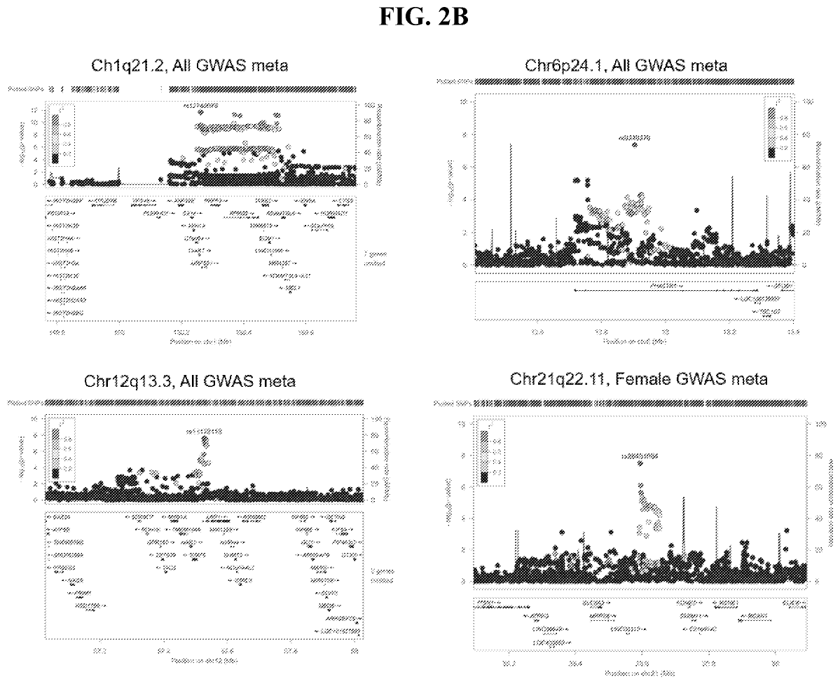

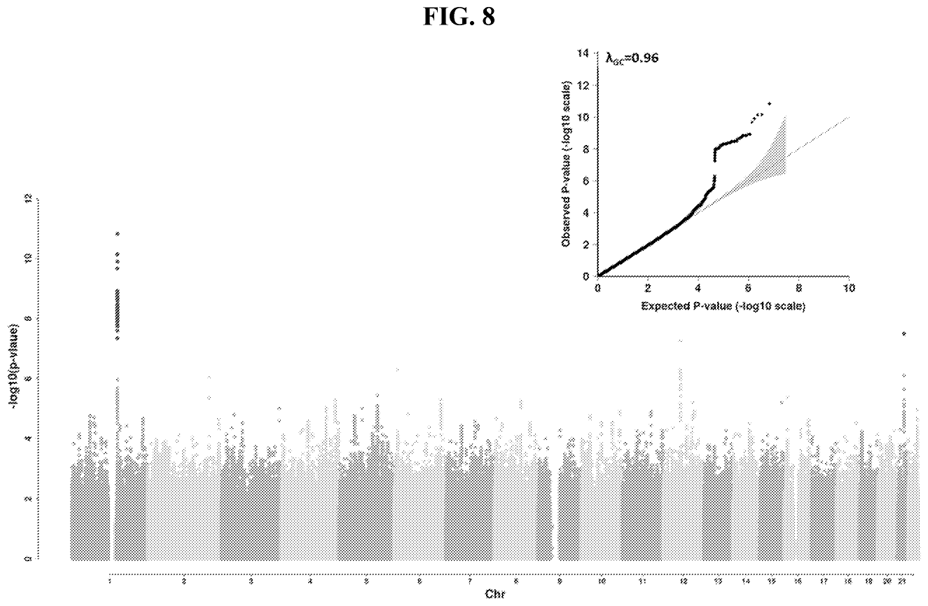

Meta-analysis of the genome-wide SCAD discovery and replication results identified additional genome-wide significant associations at the chromosome 12q13 LRP1 locus (rs11172113) and the chromosome 6p24.1 PHACTR1 locus (rs9349379) (Table 1, FIG. 2A-B, Table 3). The chromosome 6p24.1 association was consistent with a previously published association with SCAD of similar magnitude, with an OR of 1.5 in the current study compared to an OR of 1.8 (Ref. 17; incorporated by reference in its entirety). Conditional analyses based on meta-analysis of individual conditional results of discovery studies and replication studies were performed on the top loci with a false discovery rate q-value less than 0.05, which corresponded to P<5×10⁻⁶, for follow-up analysis using a polygenic risk score (PRS) approach. This review yielded seven independent loci (rs11207415, rs12740679, rs78377252, rs9349379, rs78349783, rs11172113, rs28451064) (Table 4). A secondary GWAS meta-analysis of only females in the main SCAD discovery and replication GWAS analyses (N=7,891) showed similar findings in the top chromosome 1q21.2 locus with P<5×10⁻⁸, as well as a new association at chromosome 21q22.11 (rs28451064) (OR=1.95[95% CI:1.54-2.47], P=3.2×10⁻⁸) (Table 1, FIG. 8). A secondary analysis of SCAD with and without FMD was conducted separately (N=2,922 and N=2,655, respectively), confirming that no additional signals were identified, and the results of the top three loci from the overall GWAS meta-analysis are largely consistent and noted to be ranked highly in the SCAD+FMD GWAS (Table 5). The top chromosome 1q21.2 association results in the main GWAS and secondary analyses demonstrated association of SCAD with this locus regardless of FMD status (Table 6), although not meeting a genome wide significance threshold of P<5×10⁻⁸, likely due to reduced sample size and loss of statistical power of these analyses. The discovery stage genotypes were used to estimate the heritability of SCAD, and the estimated genetic proportion of variance explained was 0.26 (standard error 0.045).

### Transcript Expression Analyses for Quantitative Trait Loci and Sex Differences

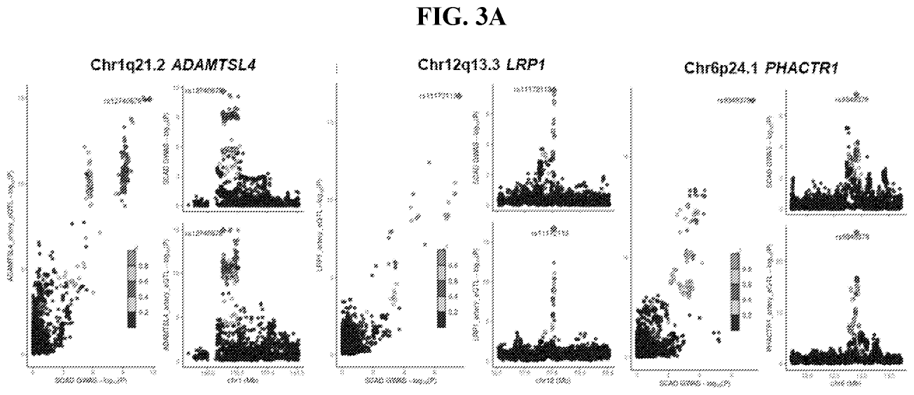

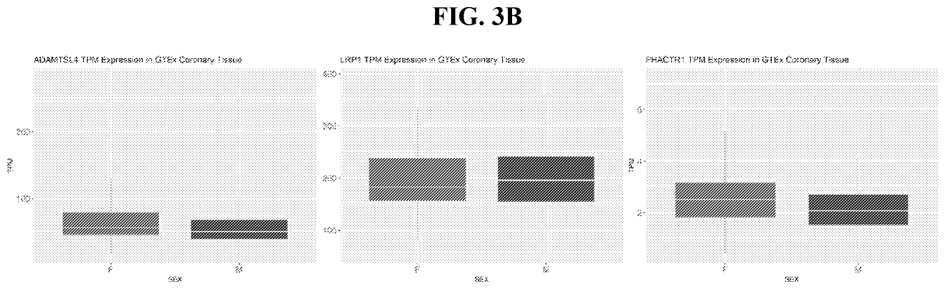

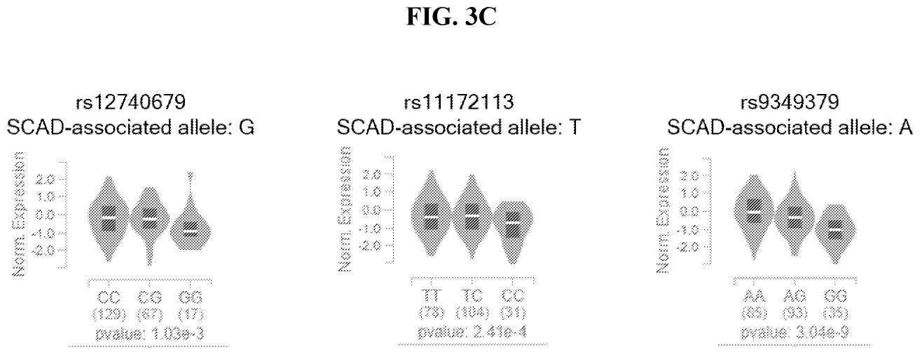

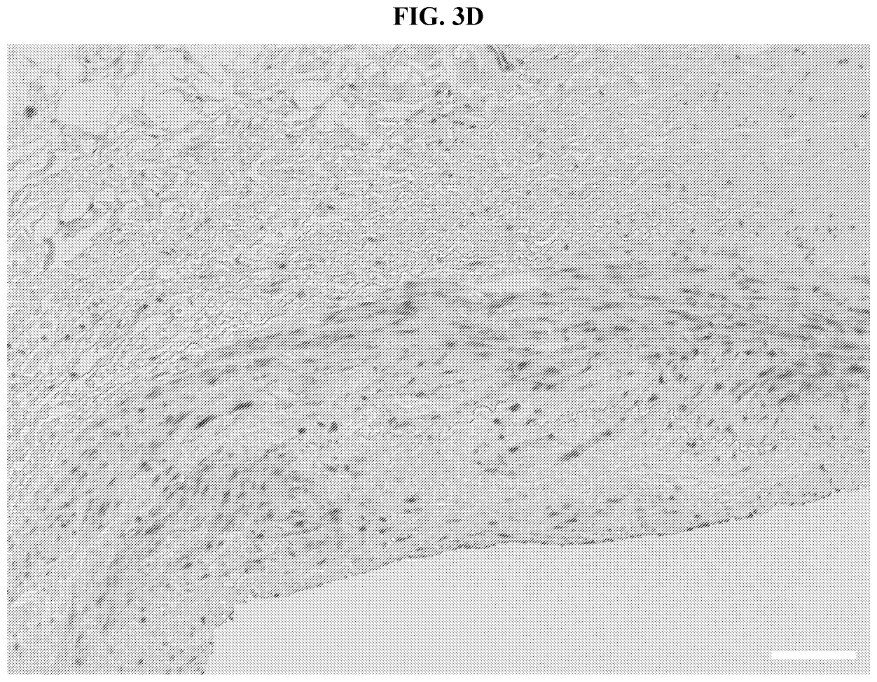

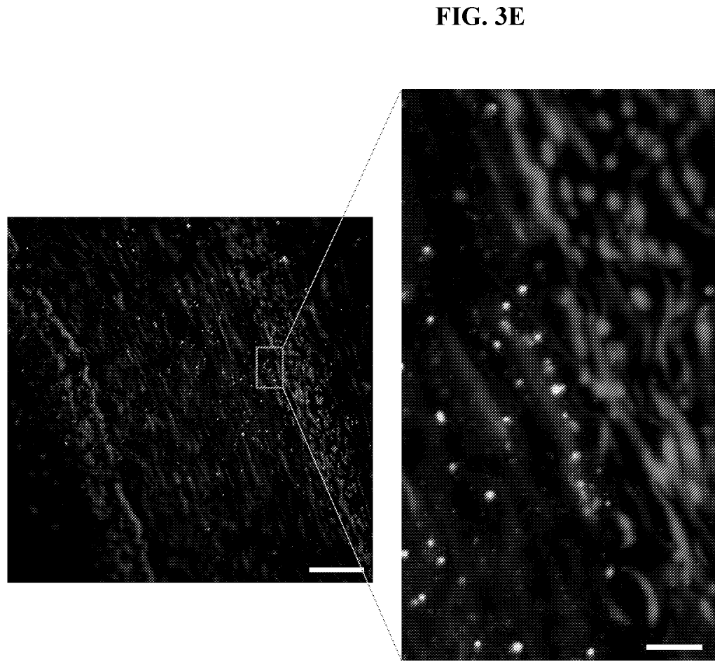

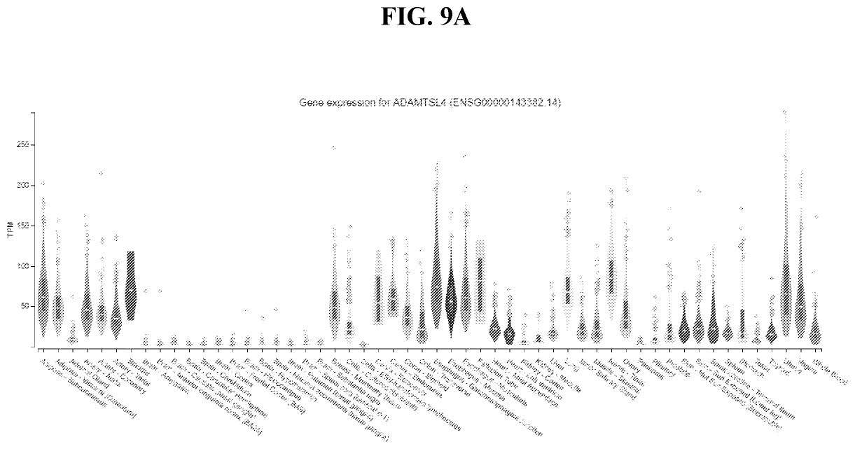

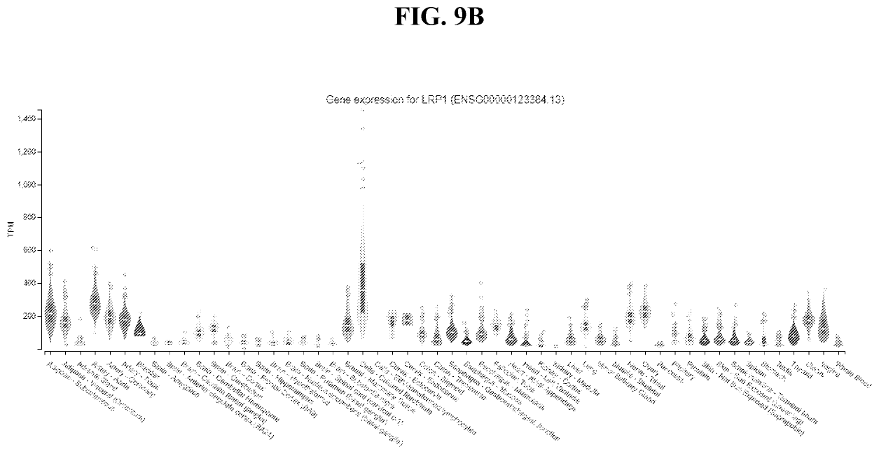

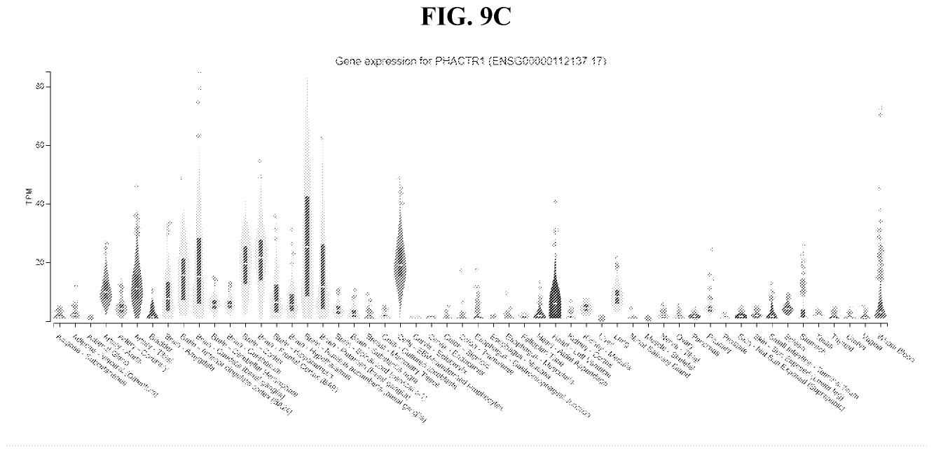

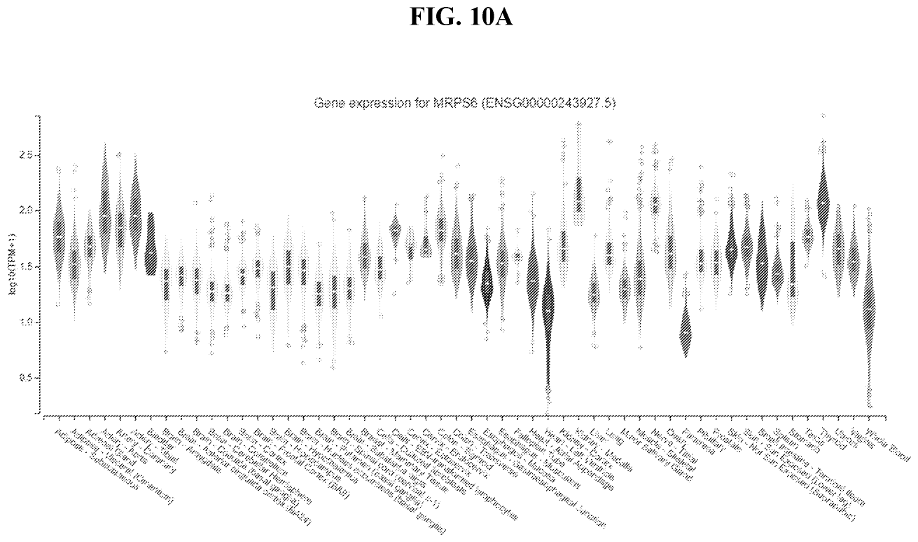

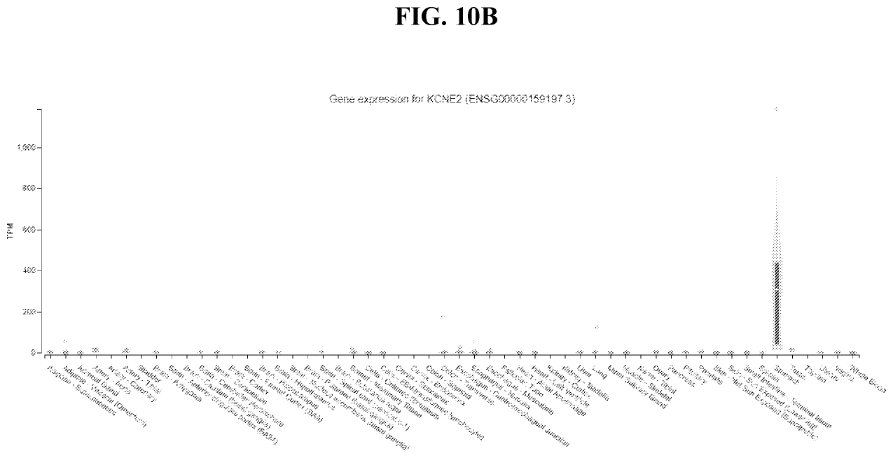

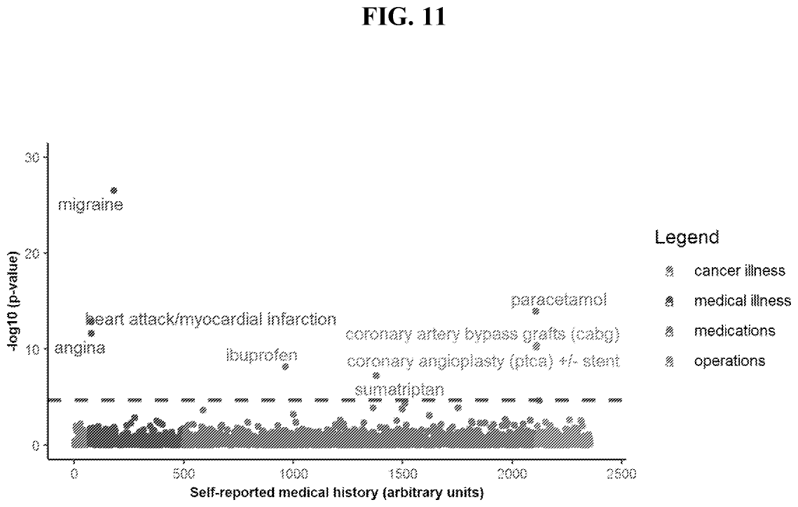

It was contemplated that the functional gene(s) at the chromosome 1q21.2 locus would have expression levels regulated by the index variant rs12740679 as an expression quantitative trait locus (eQTL) that would be identified in a colocalization analysis, and that the regulated gene(s) would be expressed in vascular tissue and smooth muscle cells. The Genotype-Tissue Expression (GTEx) portal (Ref 28; incorporated by reference in its entirety) was used to examine the mRNA expression of genes within the 500 kb surrounding the noncoding rs12740679 locus and identified the most significant association in the colocalization analysis in arterial tissues (coronary artery, tibial artery, and aorta) for ADAMTSL4 (FIG. 3a), with eQTL linear regression Pₐₒᵣₜₐ=2.6×10⁻¹⁷ and P_{tibial artery}=2.0×10⁻²⁵ (Table 7). Coronary artery expression demonstrated wider variability in expression value, with an eQTL association of P_{coronary}=1.03×10⁻³ (FIG. 3b). ADAMTSL4 mRNA was strongly expressed in arterial tissues, as well as other organs comprised of smooth muscle (FIGS. 9A-C). In order to localize ADAMTSL4 protein and mRNA expression, immunostaining and in situ hybridization were performed (FIG. 3d,e); these studies both demonstrated expression in the arterial media smooth muscle cells, consistent with the location of arterial disruption in SCAD. The expression of ADAMTSL4 in arterial tissues was 1.1-fold higher in women as compared to men across all arterial tissues in GTEx (Wald test P=1.3×10⁻⁵) (FIG. 3b, Table 7). In the chromosomes 6p24.1 and 12q13.3 loci, rs9349379 and rs11172113 were each identified as significant eQTLs in arterial tissues regulating the expression of PHACTR1 and LRPJ, respectively (FIG. 3a-c, Table 7), with PHACTR1 mRNA expressed 1.2-fold higher in the coronary arteries of women (Wald test P=8.2×10⁻³) (Table 7). In the chromosome 21q22.11 locus, rs28451064, although no genes passed the threshold of 75% posterior probability, MRP6 and KCNE2 were identified as the top transcripts in the colocalization analysis. MRP6 is expressed in arteries (Table 7, FIGS. 10A-B) but showed no differences in expression level according to sex (Table 7).

### Analysis of a SCAD Polygenic Risk Score (PRS_{SCAD}) in an At-Risk FMD Cohort

Arterial dissections occur in a subset of individuals with multifocal FMD (mFMD) and may lead to substantial morbidity due to organ hypoperfusion and ischemia, with clinical manifestations including MI due to SCAD. Given the overlap of FMD and SCAD diagnoses in some individuals, it was contemplated that alleles discovered by GWAS would be associated with SCAD events in individuals with mFMD and tested this in a cohort of individuals with mFMD consecutively enrolled at presentation to subspecialty clinics with angiographic evidence of mFMD. As a confirmation analysis of the chromosome 1q21.2 association with SCAD, a case-control GWAS was performed using the FMD cohort cases and age-, sex- and ethnicity-matched controls from the Cleveland Clinic Genebank, which showed replication at this locus (OR=1.92[95% CI:1.06-3.45], P=0.032, GWAS λ_{OC}=0.98). Next, it was tested whether the top-ranked alleles identified in the SCAD GWAS meta-analysis were predictive of SCAD in the FMD cohort. Among the 412 unrelated subjects 131 subjects had at least one arterial dissection, and 281 subjects had no documented arterial dissections; 28 unrelated individuals had experienced SCAD. Applying a PRS developed from the top-ranked SCAD GWAS meta-analysis loci with false discovery rate q value<0.05 in the SCAD meta-analysis (PRS_{SCAD}), the PRS_{SCAD} was associated with increased SCAD risk in a weighted continuous logistic regression model (OR=1.82 per 1 SD unit, 95% CI [1.09-3.02], P=0.021, Table 8). The PRS_{SCAD} result is driven primarily by an association of rs11207415 on chromosome 1p32.1 (OR=2.09[95% CI:1.21-3.63], P=9.36×10⁻³,) that is independent of the top SCAD-associated variant, rs12740679 at chromosome 1q21.2 (Table 9). A vascular phenome-wide association study (“vascular PheWAS”) was conducted as a secondary analysis in the FMD samples fully independent of the SCAD GWAS meta-analysis, to comprehensively assess the occurrence of arterial aneurysm, dissection and stenotic lesions of FMD, by testing the association of the PRS_{SCAD} with each vascular finding using a continuous logistic regression (Table 8), showing nominal associations (P<0.05) with cervical artery mFMD (OR=1.58[95% CI:1.13-2.22] per 1 SD unit, P=0.0079) and an inverse association with hypertension (OR=0.74[95% CI:0.57-0.97] per 1 SD unit, P=0.029) (Table 8). These results indicate that the alleles in the PRS_{SCAD} predispose to fragility of the coronary arterial wall, even among those already affected with FMD.

### Pleiotropy of the SCAD-Associated Risk Loci

The chromosome 6p24.1 PHACTR1 locus rs9349379-A allele has been associated with SCAD, FMD, cervical artery dissection, migraine headache, and hypertension, and the rs9349379-G allele has been associated with coronary artery disease (CAD) and MI more typically due to atherothrombotic mechanisms and more frequently occurring in men (Refs. 12, 17-18, 29-30; incorporated by reference in their entireties). All three of the discovered loci in the SCAD GWAS meta-analysis have been described in association with migraine headache, which is observed in 32.3% of patients with FMD (Ref 31; incorporated by reference in its entirety) and 32.9% of the CanSCAD cohort (Table 2, Table 10). Using risk scores based upon CAD-associated SNPs (386 SNPs derived from the GWAS Catalog), a strong inverse relationship was observed between the score, with genetically increased CAD risk conferring a protective effect from SCAD, (OR=0.78 per 1 SD unit increase, 95% CI [0.68-0.89], P=3.23×10⁻⁴), and this was robust to the removal of the chromosome 6p24.1 locus from the risk score (OR=0.82 per 1 SD unit increase, 95% CI:0.71-0.94, P=3.75×10⁻³) with several individual SNPs showing nominal association (P<0.05) with opposing direction of effect between CAD and SCAD (Table 11, Table 12).

### Analysis of the PRS_{SCAD} in the UK Biobank and Million Veteran Program

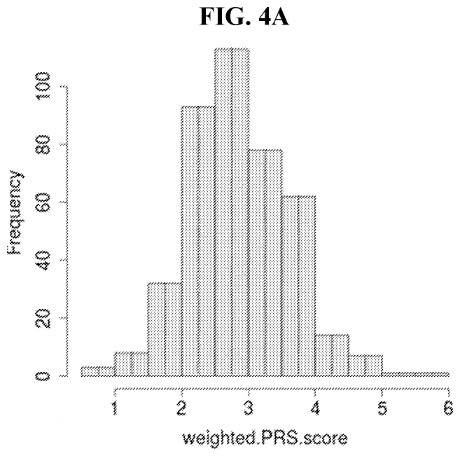

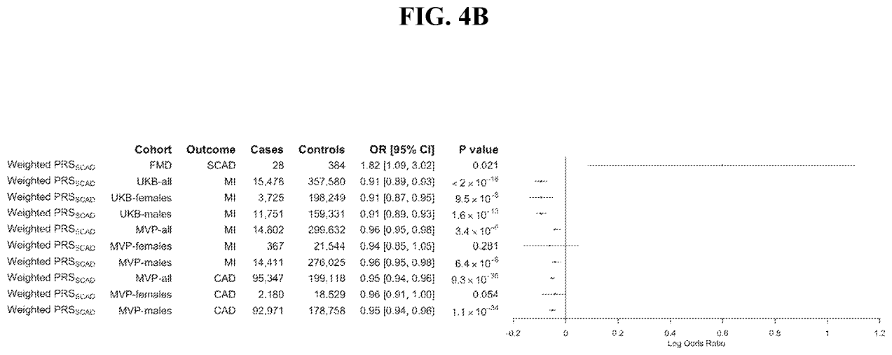

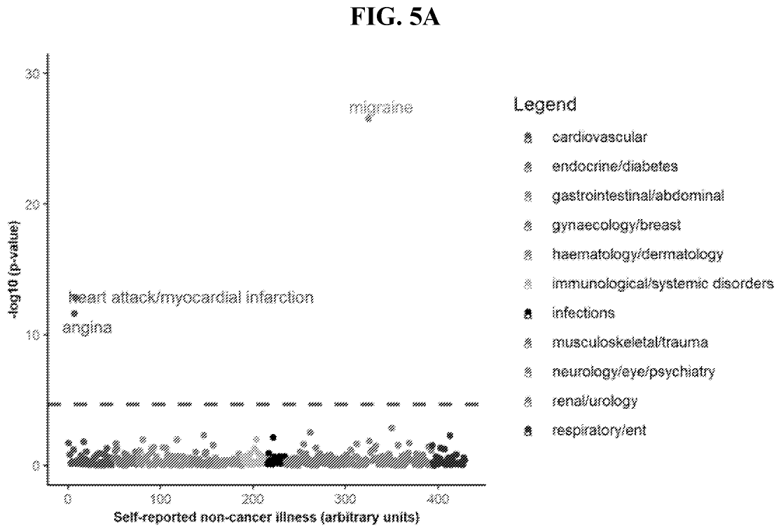

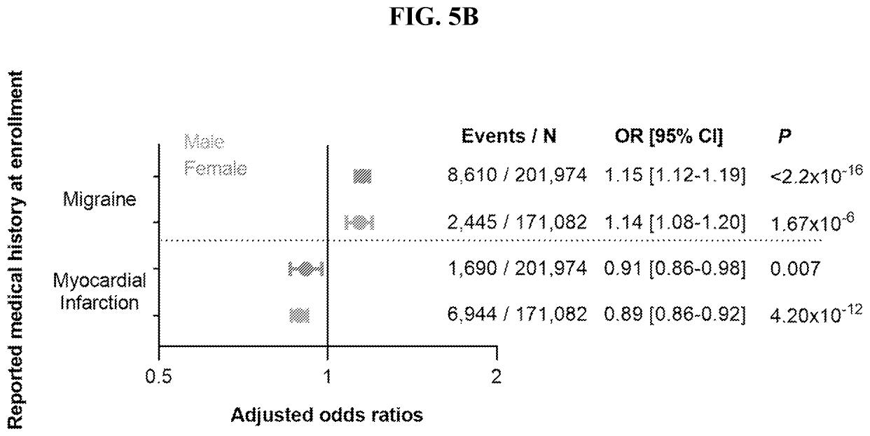

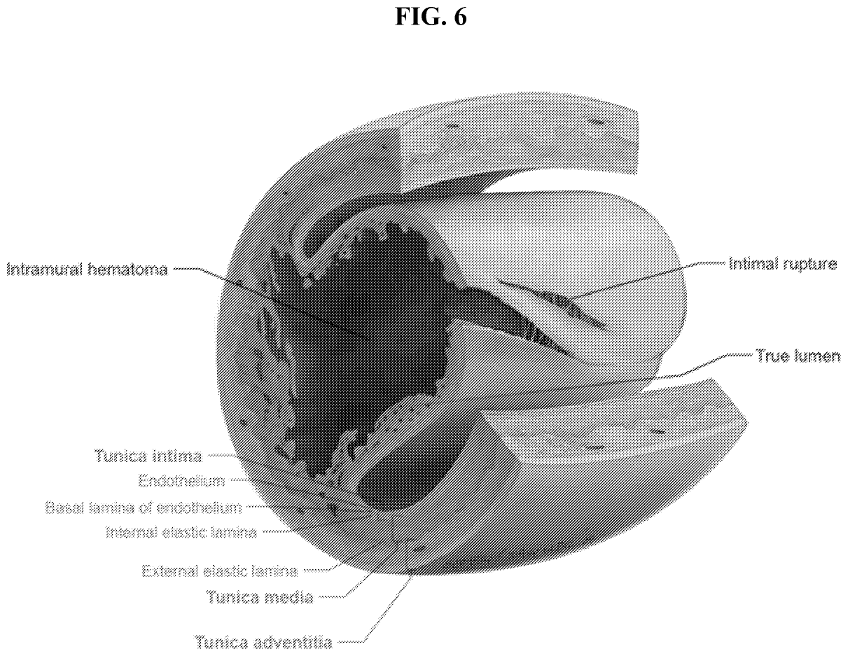

Pleiotropy of the SCAD-associated loci in the UKB published results (geneatlas.roslin.ed.ac.uk/phewas/PheWAS) was further assessed, demonstrating associations of chr6p24.1 rs9349379-G (PHACTR1) and chr21q22.11 rs28451064-A (MRP6/KCNE2) with CAD (Table 10). There was insufficient phenotyping and/or too few events of SCAD and MI in women under the age of 50 years, to replicate the associations with SCAD in the UKB. Of the SNPs included in the PRS_{SCAD}, two SNPs had at least nominal association with atherosclerotic-MI risk (Table 9) at chromosome 6p24.1 (rs9349379) and chromosome 21q22.11 (rs28451064), with each locus having a directionally opposite effect for SCAD. The effect estimate of the inverse association of the PRS_{SCAD} with MI was comparable between men (adjusted HR=0.91 per 1 SD unit increase, 95% CI [0.89-0.93], P=1.57×10⁻¹³) and women (adjusted HR=0.91 per 1 SD unit increase, 95% CI [0.87-0.95], P=9.46×10⁻⁶; FIG. 4A-B, Table 13), with a 3.72-fold higher MI event-rate in men (N=11,751/ 171,082) as compared to women (N=3,725/ 201,974). There was no significant sex interaction with the PRS_{SCAD}. The inverse association of the PRS_{SCAD} with CAD risk (OR=0.95 per 1 SD unit increase, 95% CI [0.94-0.96], P=9.33×10⁻³⁶) and MI risk (OR=0.96 per 1 SD unit increase, 95% CI [0.95-0.98], P=3.35×10⁻⁶) was replicated in the Millions Veteran Program (MVP), and also without substantial differences in effect estimates between men and women (Table 14). Both the UKB and MVP analyses were repeated after removing the chromosome 6p24.1 locus already known to have inverse risk for CAD/MI and SCAD, and the results remained comparable and statistically significant (Tables 13 and 14). Thus, the UKB and MVP results both robustly support that there are opposing mechanisms between SCAD-related MI and atherosclerotic MI/CAD, involving the top-ranked loci identified in our SCAD GWAS meta-analysis (FIG. 4B, Table 12). A PheWAS of UKB data highlighted that a weighted and continuous PRS_{SCAD} was associated with MI, migraine headache, medications used to treat migraine headache, tinnitus, and coronary artery revascularization, with concordant odds ratios in men and women for both migraine headache and MI (FIGS. 5A-B, FIG. 7, Table 15).

### TABLES

Lengthy table referenced here
US20230220472A1-20230713-T00001
Please refer to the end of the specification for access instructions.

Lengthy table referenced here
US20230220472A1-20230713-T00002
Please refer to the end of the specification for access instructions.

Lengthy table referenced here
US20230220472A1-20230713-T00003
Please refer to the end of the specification for access instructions.

Lengthy table referenced here
US20230220472A1-20230713-T00004
Please refer to the end of the specification for access instructions.

Lengthy table referenced here
US20230220472A1-20230713-T00005
Please refer to the end of the specification for access instructions.

Lengthy table referenced here
US20230220472A1-20230713-T00006
Please refer to the end of the specification for access instructions.

Lengthy table referenced here
US20230220472A1-20230713-T00007
Please refer to the end of the specification for access instructions.

Lengthy table referenced here
US20230220472A1-20230713-T00008
Please refer to the end of the specification for access instructions.

Lengthy table referenced here
US20230220472A1-20230713-T00009
Please refer to the end of the specification for access instructions.

Lengthy table referenced here
US20230220472A1-20230713-T00010
Please refer to the end of the specification for access instructions.

Lengthy table referenced here
US20230220472A1-20230713-T00011
Please refer to the end of the specification for access instructions.

Lengthy table referenced here
US20230220472A1-20230713-T00012
Please refer to the end of the specification for access instructions.

Lengthy table referenced here
US20230220472A1-20230713-T00013
Please refer to the end of the specification for access instructions.

Lengthy table referenced here
US20230220472A1-20230713-T00014
Please refer to the end of the specification for access instructions.

### REFERENCES

The following references, some of which are cited above by number, are herein incorporated by reference in their entireties
1. Hayes, S. N. et al. Spontaneous Coronary Artery Dissection: Current State of the Science: A Scientific Statement From the American Heart Association. Circulation 137, e523-e557 (2018).
2. Saw, J., Mancini, G. B. J. & Humphries, K. H. Contemporary Review on Spontaneous Coronary Artery Dissection. J Am Coll Cardiol 68, 297-312 (2016).
3. Saw, J., Mancini, G. B. & Humphries, K. H. Contemporary Review on Spontaneous Coronary Artery Dissection. J Am Coll Cardiol 68, 297-312 (2016).
4. Brodsky, S. V., Ramaswamy, G., Chander, P. & Braun, A. Ruptured cerebral aneurysm and acute coronary artery dissection in the setting of multivascular fibromuscular dysplasia: a case report. Angiology 58, 764-7 (2007).
5. Lie, J. T. & Berg, K. K. Isolated fibromuscular dysplasia of the coronary arteries with spontaneous dissection and myocardial infarction. Hum Pathol 18, 654-6 (1987).
6. Mather, P. J. et al. Postpartum multivessel coronary dissection. J Heart Lung Transplant 13, 533-7 (1994).
7. Moulson, N., Kelly, J., Iqbal, M. B. & Saw, J. Histopathology of Coronary Fibromuscular Dysplasia Causing Spontaneous Coronary Artery Dissection. JACC Cardiovasc Intery 11, 909-910 (2018).
8. Saw, J. et al. Spontaneous Coronary Artery Dissection: Clinical Outcomes and Risk of Recurrence. J Am Coll Cardiol 70, 1148-1158 (2017).
9. Saw, J., Ricci, D., Starovoytov, A., Fox, R. & Buller, C. E. Spontaneous coronary artery dissection: prevalence of predisposing conditions including fibromuscular dysplasia in a tertiary center cohort. JACC Cardiovasc Intery 6, 44-52 (2013).
10. Kadian-Dodov, D. et al. Dissection and Aneurysm in Patients With Fibromuscular Dysplasia: Findings From the U.S. Registry for FMD. J Am Coll Cardiol 68, 176-85 (2016).
11. Pannier-Moreau, I. et al. Possible familial origin of multifocal renal artery fibromuscular dysplasia. J Hypertens 15, 1797-801 (1997).
12. Perdu, J. et al. Inheritance of arterial lesions in renal fibromuscular dysplasia. J Hum Hypertens 21, 393-400 (2007).
13. Rushton, A. R. The genetics of fibromuscular dysplasia. Arch Intern Med 140, 233-6 (1980).
14. Kiando, S. R. et al. PHACTR1 Is a Genetic Susceptibility Locus for Fibromuscular Dysplasia Supporting Its Complex Genetic Pattern of Inheritance. PLoS Genet 12, e1006367 (2016).
15. Goel, K. et al. Familial spontaneous coronary artery dissection: evidence for genetic susceptibility. JAMA Intern Med 175, 821-6 (2015).
16. Turley, T. N. et al. Rare Missense Variants in TLN1 Are Associated With Familial and Sporadic Spontaneous Coronary Artery Dissection. Circ Genom Precis Med 12, e002437 (2019).
17. Adlam, D. et al. Association of the PHACTR1/EDN1 Genetic Locus With Spontaneous Coronary Artery Dissection. J Am Coll Cardiol 73, 58-66 (2019).
18. Debette, S. et al. Common variation in PHACTR1 is associated with susceptibility to cervical artery dissection. Nat Genet 47, 78-83 (2015).
19. Gormley, P. et al. Meta-analysis of 375,000 individuals identifies 38 susceptibility loci for migraine. Nat Genet 48, 856-66 (2016).
20. Anttila, V. et al. Genome-wide meta-analysis identifies new susceptibility loci for migraine. Nat Genet 45, 912-917 (2013).
21. Freilinger, T. et al. Genome-wide association analysis identifies susceptibility loci for migraine without aura. Nat Genet 44, 777-82 (2012).
22. Henkin, S. et al. Spontaneous coronary artery dissection and its association with heritable connective tissue disorders. Heart (2016).
23. Kaadan, M. I. et al. Prospective Cardiovascular Genetics Evaluation in Spontaneous Coronary Artery Dissection.Circ Genom Precis Med 11, e001933 (2018).
24. Gornik, H. L. et al. First International Consensus on the diagnosis and management of fibromuscular dysplasia. Vasc Med 24, 164-189 (2019).
25. Saw, J. et al. Canadian spontaneous coronary artery dissection cohort study: in-hospital and 30-day outcomes. Eur Heart J (2019).
26. Zhou, W. et al. Efficiently controlling for case-control imbalance and sample relatedness in large-scale genetic association studies. Nat Genet 50, 1335-1341 (2018).
27. McCarthy, S. et al. A reference panel of 64,976 haplotypes for genotype imputation. Nat Genet 48, 1279-83 (2016).
28. Consortium, G. T. The Genotype-Tissue Expression (GTEx) project. Nat Genet 45, 580-5 (2013).
29. Nikpay, M. et al. A comprehensive 1,000 Genomes-based genome-wide association meta-analysis of coronary artery disease. Nat Genet 47, 1121-1130 (2015).
30. Surendran, P. et al. Trans-ancestry meta-analyses identify rare and common variants associated with blood pressure and hypertension. Nat Genet 48, 1151-1161 (2016).
31. Olin, J. W. et al. The United States Registry for Fibromuscular Dysplasia: results in the first 447 patients. Circulation 125, 3182-90 (2012).
32. Buniello, A. et al. The NHGRI-EBI GWAS Catalog of published genome-wide association studies, targeted arrays and summary statistics 2019. Nucleic Acids Res 47, D1005-D1012 (2019).
33. Canela-Xandri, O., Rawlik, K. & Tenesa, A. An atlas of genetic associations in UK Biobank. Nat Genet 50, 1593-1599 (2018).
34. Saw, J., Bezerra, H., Gornik, H. L., Machan, L. & Mancini, G. B. Angiographic and Intracoronary Manifestations of Coronary Fibromuscular Dysplasia. Circulation (2016).
35. Hubmacher, D. & Apte, S. S. ADAMTS proteins as modulators of microfibril formation and function. Matrix Biol 47, 34-43 (2015).
36. Chandra, A. et al. A genotype-phenotype comparison of ADAMTSL4 and FBN1 in isolated ectopia lentis. Invest Ophthalmol Vis Sci 53, 4889-96 (2012).
37. Collin, G. B. et al. Disruption of murine Adamtsl4 results in zonular fiber detachment from the lens and in retinal pigment epithelium dedifferentiation. Hum Mol Genet 24, 6958-74 (2015).
38. Li, J., Jia, X., Li, S., Fang, S. & Guo, X. Mutation survey of candidate genes in 40 Chinese patients with congenital ectopia lentis. Mol Vis 20, 1017-24 (2014).
39. Dietz, H. C. et al. Marfan syndrome caused by a recurrent de novo missense mutation in the fibrillin gene. Nature 352, 337-9 (1991).
40. Dietz, H. C. & Pyeritz, R. E. Mutations in the human gene for fibrillin-1 (FBN1) in the Marfan syndrome and related disorders. Hum Mol Genet 4 Spec No, 1799-809 (1995).
41. Neptune, E. R. et al. Dysregulation of TGF-beta activation contributes to pathogenesis in Marfan syndrome. Nat Genet 33, 407-11 (2003).
42. Wang, X. & Musunuru, K. Confirmation of Causal rs9349379-PHACTR1 Expression Quantitative Trait Locus in Human-Induced Pluripotent Stem Cell Endothelial Cells. Circ Genom Precis Med 11, e002327 (2018).
43. Gupta, R. M. et al. A Genetic Variant Associated with Five Vascular Diseases Is a Distal Regulator of Endothelin-1 Gene Expression. Cell 170, 522-533 e15 (2017).
44. Bown, M. J. et al. Abdominal aortic aneurysm is associated with a variant in low-density lipoprotein receptor-related protein 1. Am J Hum Genet 89, 619-27 (2011).
45. Boucher, P., Gotthardt, M., Li, W. P., Anderson, R. G. & Herz, J. LRP: role in vascular wall integrity and protection from atherosclerosis. Science 300, 329-32 (2003).
46. Lee, C. C. et al. Risk of Aortic Dissection and Aortic Aneurysm in Patients Taking Oral Fluoroquinolone. JAMA Intern Med 175, 1839-47 (2015).
47. Saw, J., Starovoytov, A., Zhao, Y., Peng, D. & Humphries, K. Clinical predictors of recurrent spontaneous coronary artery dissection. JACC 69, 273 (2017).
48. Doyle, J. J. et al. A deleterious gene-by-environment interaction imposed by calcium channel blockers in Marfan syndrome. Elife 4(2015).
49. Ntalla, I. et al. Genetic Risk Score for Coronary Disease Identifies Predispositions to Cardiovascular and Noncardiovascular Diseases. J Am Coll Cardiol 73, 2932-2942 (2019).
50. Daghlas, I., Guo, Y. & Chasman, D. I. Effect of genetic liability to migraine on coronary artery disease and atrial fibrillation: a Mendelian randomization study. Eur J Neurol (2019).
51. Saw, J. et al. Angiographic appearance of spontaneous coronary artery dissection with intramural hematoma proven on intracoronary imaging. Catheter Cardiovasc Intery 87, E54-61 (2016).
52. Saw, J. Coronary angiogram classification of spontaneous coronary artery dissection. Catheter Cardiovasc Intery 84, 1115-22 (2014).
53. Sadananda, S. N. et al. Targeted next-generation sequencing to diagnose disorders of HDL cholesterol. J Lipid Res 56, 1993-2001 (2015).
54. Fritsche, L. G. et al. Association of Polygenic Risk Scores for Multiple Cancers in a Phenome-wide Study: Results from The Michigan Genomics Initiative. Am J Hum Genet 102, 1048-1061 (2018).
55. Bycroft, C. et al. The UK Biobank resource with deep phenotyping and genomic data. Nature 562, 203-209 (2018).
56. Gaziano, J. M. et al. Million Veteran Program: A mega-biobank to study genetic influences on health and disease. J Clin Epidemiol 70, 214-23 (2016).
57. Fang, H. et al. Harmonizing Genetic Ancestry and Self-identified Race/Ethnicity in Genome-wide Association Studies. Am J Hum Genet 105, 763-772 (2019).
58. Hunter-Zinck, H. et al. Measuring genetic variation in the multi-ethnic Million Veteran Program (MVP). bioRxiv, 2020.01.06.896613 (2020).
59. Purcell, S. et al. PLINK: a tool set for whole-genome association and population-based linkage analyses. Am J Hum Genet 81, 559-75 (2007).
60. Das, S. et al. Next-generation genotype imputation service and methods. Nat Genet 48, 1284-1287 (2016).
61. Li, J. Z. et al. Worldwide human relationships inferred from genome-wide patterns of variation. Science 319, 1100-4 (2008).
62. Taliun, D. et al. LASER server: ancestry tracing with genotypes or sequence reads. Bioinformatics 33, 2056-2058 (2017).
63. Wang, C., Zhan, X., Liang, L., Abecasis, G. R. & Lin, X. Improved ancestry estimation for both genotyping and sequencing data using projection procrustes analysis and genotype imputation. Am J Hum Genet 96, 926-37 (2015).
64. Willer, C. J., Li, Y. & Abecasis, G. R. METAL: fast and efficient meta-analysis of genomewide association scans. Bioinformatics 26, 2190-1 (2010).
65. Pruim, R. J. et al. LocusZoom: regional visualization of genome-wide association scan results. Bioinformatics 26, 2336-7 (2010).
66. Zhou, X. A Unified Framework for Variance Component Estimation with Summary Statistics in Genome-Wide Association Studies. Ann Appl Stat 11, 2027-2051 (2017).
67. Giambartolomei, C. et al. Bayesian test for colocalisation between pairs of genetic association studies using summary statistics. PLoS Genet 10, e1004383 (2014).
68. Boxiang Liu, M. G., Stephen Montgomery LocusCompare: A Tool to Visualize Pairs of Association. ((2018)).
69. Storey J D, B. A., Dabney A, Robinson D. qvalue: Q-value estimation for false discovery rate control. ((2019)).
70. Machiela, M. J. & Chanock, S. J. LDlink: a web-based application for exploring population-specific haplotype structure and linking correlated alleles of possible functional variants. Bioinformatics 31, 3555-7 (2015).
71. Millard, L. A. C., Davies, N. M., Gaunt, T. R., Davey Smith, G. & Tilling, K. Software Application Profile: PHESANT: a tool for performing automated phenome scans in UK Biobank. Int J Epidemiol (2017).
72. Aguet, F., Brown, A., Castel, S. et al. Genetic effects on gene expression across human tissues. Nature 550, 204-213 (2017).

LENGTHY TABLES
The patent application contains a lengthy table section. A copy of the table is available in electronic form from the USPTO web site (https://seqdata.uspto.gov/?pageRequest=docDetail&DocID=US20230220472A1). An electronic copy of the table will also be available from the USPTO upon request and payment of the fee set forth in 37 CFR 1.19(b)(3).

1. A method of assessing the risk of spontaneous coronary artery dissection (SCAD) in a subject suffering from fibromuscular dysplasia (FMD) comprising:
(a) testing a sample from the subject for biomarkers of SCAD; and
(b) assessing the subject's risk of SCAD.

2. The method of claim 1, wherein the biomarkers of SCAD are selected from rs11207415, rs12740679, rs78377252, rs9349379, rs78349783, rs11172113, and rs28451064.

3. The method of claim 2, wherein any combination of two or more of rs11207415, rs12740679, rs78377252, rs9349379, rs78349783, rs11172113, and rs28451064 are analyzed.

4. The method of claim 1, wherein assessing the subject's risk of SCAD comprises:
(i) calculating a risk score based on the biomarkers for SCAD; and
(ii) comparing the risk score to a threshold to determine the subject's risk for SCAD.

5. the method of claim 4, wherein the presence of any biomarker is weighted according to its odds ratio in order to calculate the risk score.

6. A method of preventing spontaneous coronary artery dissection (SCAD) in a subject suffering from fibromuscular dysplasia (FMD) comprising:
(1) assessing the risk of SCAD by the method of one of claims 1-5; and
(2) administering a prophylactic regime to reduce the subject's risk for SCAD.

7. The method of claim 6, wherein the prophylactic regime comprises one or more of:
(i) administering aspirin to the subject;
(ii) cessation of treatment with triptan medications;
(iii) administration of contraception;
(iv) avoiding activities that increase arterial strain or spikes in blood pressure;
(v) monitoring blood pressure;
(vi) administering a beta blocker; and
(vii) administering antiplatelet therapy.

8. A method of assessing the risk of atherosclerotic-related myocardial infarction (MI) in a subject comprising:
(a) testing a sample from the subject for biomarkers of that confer inverse risk for MI; and
(b) assessing the subject's risk of atherosclerotic-related MI.

9. The method of claim 8, wherein the biomarkers are selected from rs11207415, rs12740679, rs78377252, rs9349379, rs78349783, rs11172113, and rs28451064.

10. The method of claim 9, wherein any combination of two or more of rs11207415, rs12740679, rs78377252, rs9349379, rs78349783, rs11172113, and rs28451064 are analyzed.

11. The method of claim 8, wherein assessing the subject's risk of atherosclerotic-related MI comprises:
(i) calculating a risk score based on the biomarkers; and
(ii) comparing the risk score to a threshold to determine the subject's risk for atherosclerotic-related MI.

12. The method of claim 11, wherein the presence of any biomarker is weighted according to its odds ratio in order to calculate the risk score.
